(19) United States
(12) Patent Application Publication
(10) Pub. No.: US 2026/0284342 A1
(43) Pub. Date: Sep. 24, 2026

(54) CPAP APPARATUS, UPDATE SYSTEM OF CPAP SETTING DATA, UPDATE PROGRAM OF CPAP SETTING DATA, AND UPDATE METHOD OF CPAP SETTING DATA

(71) Applicant: Murata Manufacturing Co., Ltd., Nagaokakyo-shi (JP)

(72) Inventors: Hiroaki MIEDA, Nagaokakyo-shi (JP); Takemasa MURATA, Nagaokakyo-shi (JP); Kenya HASEGAWA, Nagaokakyo-shi (JP)

(21) Appl. No.: 19/554,738

(22) Filed: Mar. 3, 2026

(30) Foreign Application Priority Data
Mar. 24, 2025 (JP) 2025-048391

(51) Int. Cl. A61M 16/00; G16H 40/40

(52) U.S. Cl. CPC A61M 16/024; G16H 40/40

(57) ABSTRACT

A CPAP apparatus includes a storage device and an execution device. The execution device performs a storage process to store a reset timing when setting data has been reset in the storage device as reset timing data. The execution device acquires creation timing data indicating a creation timing of the setting data to be acquired in update in response to a request for the update of the setting data. The execution device determines whether a specific condition is met. The specific condition includes a requirement in which the reset timing indicated by the reset timing data is before the creation timing indicated by the creation timing data. The execution device updates the setting data stored in the storage device to the setting data associated with the creation timing data if it is determined that the specific condition is met.

### CROSS REFERENCE TO RELATED APPLICATION

This application claims priority from Japanese Patent Application No. 2025-048391, filed on Mar. 24, 2025. The content of this applications is incorporated herein by reference in its entirety.

### BACKGROUND OF THE DISCLOSURE

### 1. Field of the Disclosure

The present disclosure relates to a continuous positive airway pressure (CPAP) apparatus, an update system of CPAP setting data, an update program of the CPAP setting data, and an update method of the CPAP setting data.

### 2. Description of the Related Art

A CPAP apparatus in International Publication No. 2024/116638 includes a mask, a hose, a fan, and a control unit. The mask is worn by a user of the CPAP apparatus, that is, a patient using the CPAP apparatus so that the nose or the mouth of the patient is covered with the mask. The fan is connected to the mask via the hose. The fan is capable of pressure-feeding air taken by the fan to the mask via the hose. The control unit supplies a control signal to the fan to control the operation of the fan.

### BRIEF SUMMARY OF THE DISCLOSURE

The CPAP apparatus is used in, for example, the following situation. It is assumed that, when a certain patient visits a medical facility, a doctor in the medical facility diagnoses necessity of treatment using the CPAP apparatus for the patient. In this case, the patient borrows the CPAP apparatus from the medical facility to use the CPAP apparatus. After the treatment using the CPAP apparatus is terminated, the patient returns the CPAP apparatus to the medical facility. Accordingly, after a first patient used the CPAP apparatus, a second patient different from the first patient may use the CPAP apparatus. Instead of the borrowing of the CPAP apparatus from the medical facility by the patient in the above manner, the patient may buy the CPAP apparatus from the medical facility to use the CPAP apparatus. Also in this situation, when the patient buys a secondhand CPAP apparatus, the second patient may use the CPAP apparatus after the first patient uses the CPAP apparatus.

The control unit in the CPAP apparatus generally stores setting data concerning the operation of the CPAP apparatus. In updating the setting data concerning the CPAP apparatus, for example, the setting data may be updated via a communication line or a portable storage medium after the second patient has borrowed the CPAP apparatus from the medical facility. In such update of the setting data, the setting data concerning the CPAP apparatus may be updated to the setting data about the first patient regardless of a situation in which the second patient uses the CPAP apparatus. In other words, the setting data may be updated to false setting data in the CPAP apparatus.

In order to resolve the above problem, a CPAP apparatus includes a storage device configured to store setting data and an execution device. The execution device performs a reset process to reset the setting data in response to a reset request, a storage process to store a reset timing at which the setting data has been reset in the storage device as reset timing data, a creation timing acquiring process to acquire creation timing data indicating a creation timing of the setting data to be acquired in update in response to a request for the update of the setting data, a determination process to determine whether a specific condition is met, the specific condition including a requirement in which the reset timing indicated by the reset timing data is before the creation timing indicated by the creation timing data, and an update process to update the setting data stored in the storage device to the setting data associated with the creation timing data if it is determined in the determination process that the specific condition is met.

In order to resolve the above problem, an update system of CPAP setting data includes a CPAP apparatus including a device-side storage device storing setting data and a device-side execution device and a server including a server-side storage device storing the setting data and a server-side execution device updating the setting data in the CPAP apparatus. The device-side execution device performs a reset process to reset the setting data in response to a reset request and a storage process to store a reset timing at which the setting data has been reset in the device-side storage device as reset timing data. The server-side execution device performs a creation timing acquiring process to acquire creation timing data indicating a creation timing of the setting data stored in the server-side storage device in response to a request for update of the setting data in the CPAP apparatus, a reset timing acquiring process to acquire the reset timing data stored in the device-side storage device in response to a request for update of the setting data in the CPAP apparatus, a determination process to determine whether a specific condition is met, the specific condition including a requirement in which the reset timing indicated by the reset timing data is before the creation timing indicated by the creation timing data, and an update process to transmit the setting data associated with the creation timing data to the CPAP apparatus to update the setting data stored in the device-side storage device to the setting data associated with the creation timing data if it is determined in the determination process that the specific condition is met.

In order to resolve the above problem, an update program of CPAP setting data is applied to a CPAP apparatus including a storage device storing setting data and an execution device. The update program causes the execution device to perform a reset process to reset the setting data in response to a reset request, a storage process to store a reset timing at which the setting data has been reset in the storage device as reset timing data, a creation timing acquiring process to acquire creation timing data indicating a creation timing of the setting data to be acquired in update in response to a request for the update of the setting data, a determination process to determine whether a specific condition is met, the specific condition including a requirement in which the reset timing indicated by the reset timing data is before the creation timing indicated by the creation timing data, and an update process to update the setting data stored in the storage device to the setting data associated with the creation timing data if it is determined in the determination process that the specific condition is met.

In order to resolve the above problem, an update method of CPAP setting data is applied to a CPAP apparatus including a storage device storing setting data and an execution device. The execution device performs a reset process to reset the setting data in response to a reset request, a storage process to store a reset timing at which the setting data has been reset in the storage device as reset timing data, a creation timing acquiring process to acquire creation timing data indicating a creation timing of the setting data to be acquired in update in response to a request for the update of the setting data, a determination process to determine whether a specific condition is met, the specific condition including a requirement in which the reset timing indicated by the reset timing data is before the creation timing indicated by the creation timing data, and an update process to update the setting data stored in the storage device to the setting data associated with the creation timing data if it is determined in the determination process that the specific condition is met.

In order to resolve the above problem, an update program of CPAP setting data is applied to an update system including a CPAP apparatus including a device-side storage device storing setting data and a device-side execution device and a server including a server-side storage device storing the setting data and a server-side execution device updating the setting data in the CPAP apparatus. The device-side execution device performs a reset process to reset the setting data in response to a reset request and a storage process to store a reset timing at which the setting data has been reset in the device-side storage device as reset timing data. The update program causes the server-side execution device to perform a creation timing acquiring process to acquire creation timing data indicating a creation timing of the setting data stored in the server-side storage device in response to a request for update of the setting data in the CPAP apparatus, a reset timing acquiring process to acquire the reset timing data stored in the device-side storage device in response to a request for update of the setting data in the CPAP apparatus, a determination process to determine whether a specific condition is met, the specific condition including a requirement in which the reset timing indicated by the reset timing data is before the creation timing indicated by the creation timing data, and an update process to transmit the setting data associated with the creation timing data to the CPAP apparatus to update the setting data stored in the device-side storage device to the setting data associated with the creation timing data if it is determined in the determination process that the specific condition is met.

In order to resolve the above problem, an update method of CPAP setting data is applied to an update system including a CPAP apparatus including a device-side storage device storing setting data and a device-side execution device and a server including a server-side storage device storing the setting data and a server-side execution device updating the setting data in the CPAP apparatus. The device-side execution device performs a reset process to reset the setting data in response to a reset request and a storage process to store a reset timing at which the setting data has been reset in the device-side storage device as reset timing data. The server-side execution device performs a creation timing acquiring process to acquire creation timing data indicating a creation timing of the setting data stored in the server-side storage device in response to a request for update of the setting data in the CPAP apparatus, a reset timing acquiring process to acquire the reset timing data stored in the device-side storage device in response to a request for update of the setting data in the CPAP apparatus, a determination process to determine whether a specific condition is met, the specific condition including a requirement in which the reset timing indicated by the reset timing data is before the creation timing indicated by the creation timing data, and an update process to transmit the setting data associated with the creation timing data to the CPAP apparatus to update the setting data stored in the device-side storage device to the setting data associated with the creation timing data if it is determined in the determination process that the specific condition is met.

With the above configurations, it is possible to suppress the update process to false setting data.

### BRIEF DESCRIPTION OF THE SEVERAL VIEWS OF THE DRAWINGS

FIG. 1 is a diagram illustrating a schematic configuration of an update system according to a first embodiment;

FIG. 2 is a sequence diagram indicating device registration control according to the first embodiment;

FIG. 3 is a sequence diagram indicating normal update control according to the first embodiment;

FIG. 4 is a sequence diagram indicating forced update control according to the first embodiment;

FIG. 5 is a sequence diagram indicating setting update control according to a second embodiment;

FIG. 6 is a diagram illustrating a schematic configuration of an update system according to a third embodiment;

FIG. 7 is a sequence diagram indicating correlation control according to the third embodiment;

FIG. 8 is a sequence diagram indicating normal update control according to the third embodiment;

FIG. 9 is a sequence diagram indicating forced update control according to the third embodiment; and

FIG. 10 is a sequence diagram indicating setting update control according to a fourth embodiment.

### DETAILED DESCRIPTION OF THE DISCLOSURE

### First Embodiment

### (Schematic Configuration of Update System)

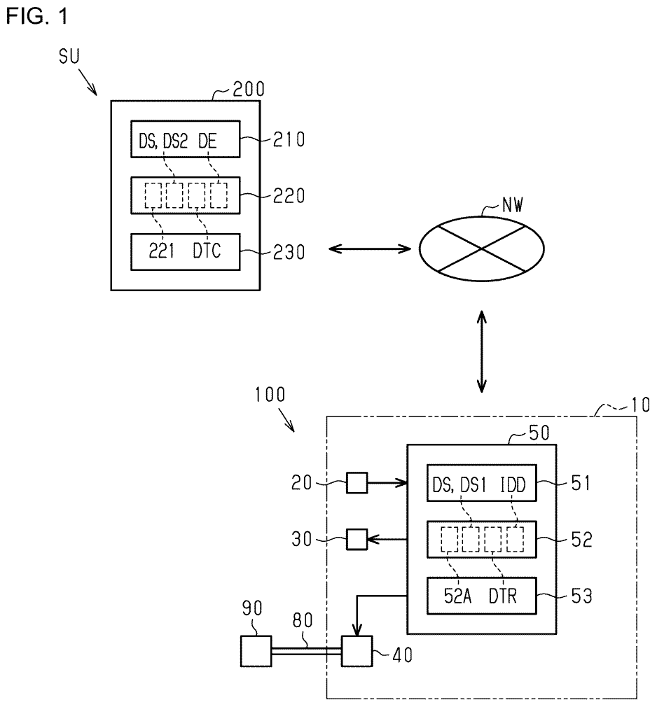

A first embodiment of the present disclosure will herein be described with reference to FIG. 1 to FIG. 4. First, a schematic configuration of an update system SU will be described.

As illustrated in FIG. 1, the update system SU includes multiple CPAP apparatuses 100. In the first embodiment, the CPAP apparatus 100 is used in, for example, the following situation. First, it is assumed that, when a certain patient visits a medical facility, a doctor in the medical facility diagnoses necessity of treatment using the CPAP apparatus for the patient. In this case, the patient borrows the CPAP apparatus from the medical facility to use the CPAP apparatus. The patient using the CPAP apparatus 100 may be referred to as a user of the CPAP apparatus 100. Only one CPAP apparatus 100 is representatively illustrated in FIG. 1.

The CPAP apparatus 100 includes a main body 10, a hose 80, and a mask 90. The main body 10 includes an operation switch 20, a display 30, a fan 40, and a control unit 50. The operation switch 20 is used by the user of the CPAP apparatus 100 to operate the CPAP apparatus 100. The display 30 is capable of displaying a variety of information.

The fan 40 includes a blower fan and an electric motor, which are not illustrated in FIG. 1. A first end of the hose 80 is connected to the fan 40. A second end of the hose 80 is connected to the mask 90. In use of the CPAP apparatus 100, the mask 90 is worn by the user so that the nose or the mouth of the user is covered with the mask 90. The fan 40 pressure-feeds air taken by the fan 40 to the mask 90 via the hose 80. In the CPAP apparatus 100, another mask 90 may be used, instead of the mask 90 attached to the main body 10. In other words, in the CPAP apparatus 100, the kind of the mask 90 may be varied depending on the size or the like of the nose or the mouth of the user of the CPAP apparatus 100.

As illustrated in FIG. 1, the control unit 50 includes an execution device 51, a storage device 52, and a communication device 53. The communication device 53 is capable of communicating with an apparatus outside the CPAP apparatus 100 via a communication network NW. An example of the execution device 51 is a central processing unit (CPU). The storage device 52 includes a readable read only memory (ROM), a readable and rewritable volatile random access memory (RAM), and a readable and rewritable non-volatile storage. The storage device 52 stores various programs and a variety of data in advance. Specifically, the storage device 52 stores a control program 52A in advance as one of the various programs. The execution device 51 executes the control program 52A to perform various processes described below. In the first embodiment, the control program 52A is an example of an update program of CPAP setting data. The execution device 51 executes the control program 52A to perform various processes concerning an update method of the CPAP setting data.

The storage device 52 stores setting data DS in advance as one piece of the various data. The setting data DS is data for settings concerning the operation of the CPAP apparatus 100. For example, the setting data DS includes an item concerning the maximum value of the pressure of the air to be supplied from the fan 40 to the mask 90, an item concerning the minimal value of the pressure of the air to be supplied from the fan 40 to the mask 90, and so on. In addition, for example, the setting data DS includes an item concerning the language to be displayed in the display 30, an item concerning turning-on and turning-off of a so-called airplane mode, and so on. When the airplane mode is turned on, the communication between the communication device 53 and an apparatus outside the CPAP apparatus 100 is blocked. The execution device 51 manages the setting data DS through the various processes described below.

The storage device 52 stores reset date and time data DTR as one piece of the variety of data. The reset date and time data DTR is data indicating the date and time when the setting data DS stored in the storage device 52 has been reset. An example of the reset date and time indicated by the reset date and time data DTR is a date and time, such as “3 p.m. on March 1st, 20XX”. The date and time indicated by initial data of the reset date and time data DTR is, for example, the date and time when the CPAP apparatus 100 has been manufactured. In the first embodiment, the reset date and time data DTR is an example of reset timing data indicating the reset timing at which the setting data DS has been reset. The resetting of the setting data DS will be described below.

The storage device 52 stores device identification data IDD as one piece of the variety of data. The device identification data IDD is data indicating information that is defined in advance for each CPAP apparatus 100 to identify the CPAP apparatus 100. An example of the device identification data IDD is a number of a dozen digits. Since the device identification data IDD is data specific to each CPAP apparatus 100, the device identification data IDD is not capable of being rewritten.

The execution device 51 in the control unit 50 supplies a control signal to the fan 40 to control the operation of the fan 40. The execution device 51 supplies a control signal to the display 30 to display a variety of information in the display 30. The control unit 50 is capable of accepting an operation by the user of the CPAP apparatus 100 with the operation switch 20.

The execution device 51 in the control unit 50 performs reset control for resetting the setting data DS. Specifically, the execution device 51 performs the reset control, for example, in the following situation. First, when the CPAP apparatus 100 is returned from the patient to the medical facility, a person in charge in the medical facility performs a predetermined operation that is defined in advance using the operation switch 20 of the CPAP apparatus 100. At this time, the execution device 51 accepts the predetermined operation to reset the setting data DS stored in the storage device 52. In addition, the execution device 51 stores the date and time when the setting data DS has been reset in the storage device 52 as the reset date and time data DTR. In other words, the execution device 51 performs a reset process to reset the setting data DS in response to a reset request. Furthermore, the execution device 51 performs a storage process to store the reset timing at which the setting data DS has been reset in the reset process in the storage device 52 as the reset timing data.

As illustrated in FIG. 1, the update system SU includes a server 200. The server 200 includes an execution device 210, a storage device 220, and a communication device 230. The communication device 230 is capable of communicating with an apparatus outside the server 200 via the communication network NW. Specifically, the communication device 230 is capable of communicating with the CPAP apparatus 100 via the communication network NW. An example of the execution device 210 is a CPU. The storage device 220 includes a readable ROM, a readable and rewritable volatile RAM, and a readable and rewritable non-volatile storage. The storage device 220 stores various programs and a variety of data in advance. Specifically, the storage device 220 stores a control program 221 in advance as one of the various programs. The execution device 210 executes the control program 221 to perform various processes described below.

The storage device 220 is capable of storing the setting data DS as one of the variety of data. The setting data DS stored in the storage device 52 in the CPAP apparatus 100 is hereinafter referred to as device-side setting data DS1 and the setting data DS stored in the storage device 220 in the server 200 is hereinafter referred to as server-side setting data DS2.

In the first embodiment, for example, the server-side setting data DS2 is created in the following situation. As described above, the user of the CPAP apparatus 100 visits the medical facility for use of the CPAP apparatus 100. After the visiting by the user of the CPAP apparatus 100, the person in charge in the medical facility creates the server-side setting data DS2 based on the content of diagnosis for the user of the CPAP apparatus 100. In response to a predetermined operation by the person in charge in the medical facility using an input device connected to the server 200, the execution device 210 in the server 200 stores the created setting data DS in the storage device 220 as the server-side setting data DS2. Accordingly, the server-side setting data DS2 may be different from the device-side setting data DS1.

In addition, the storage device 220 is capable of storing creation date and time data DTC and expiration date data DE as the variety of data. The creation date and time data DTC is data indicating the creation date and time of the server-side setting data DS2. Accordingly, in the first embodiment, the creation date and time data DTC indicates the date and time when the person in charge in the medical facility created the server-side setting data DS2. An example of the creation date and time indicated by the creation date and time data DTC is a date and time, such as “3 p.m. on April 1st, 20XX”. In the first embodiment, the creation date and time data DTC is an example of creation timing data indicating the creation timing of the server-side setting data DS2.

The expiration date data DE is data indicating the expiration date of the server-side setting data DS2. In the first embodiment, the expiration date indicated by the expiration date data DE is, for example, a point of time several months to a dozen or more months after the date and time when the person in charge in the medical facility created the server-side setting data DS2. Accordingly, an example of the expiration date indicated by the expiration date data DE is a date and time, such as “3 p.m. on October 1st, 20XX”.

### (Device Registration Control)

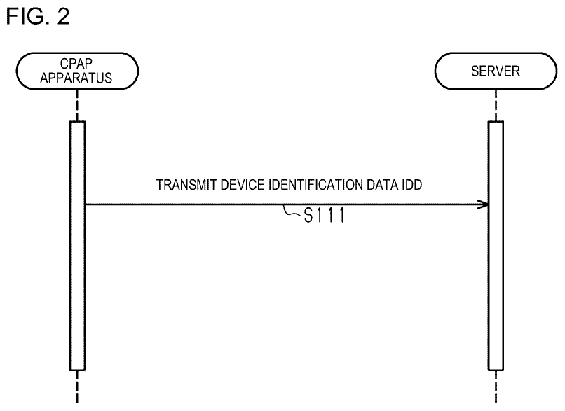

Device registration control performed by the CPAP apparatus 100 and the server 200 will now be described with reference to FIG. 2. The device registration control is the control to register the device identification data IDD about the CPAP apparatus 100 in the storage device 220 in the server 200. The device registration control may be performed in parallel between each of the multiple CPAP apparatuses 100 and the server 200. In the first embodiment, for example, when the medical facility has bought the CPAP apparatus 100, in response to a predetermined operation by the person in charge in the medical facility using the operation switch 20 of the CPAP apparatus 100, the execution device 51 in the CPAP apparatus 100 starts the device registration control. Accordingly, in the first embodiment, it is sufficient for the device registration control to be performed between the target CPAP apparatus 100 and the server 200 only once.

As illustrated in FIG. 2, at the start of the device registration control, the execution device 51 in the CPAP apparatus 100 performs Step S111. In Step S111, the execution device 51 in the CPAP apparatus 100 transmits the device identification data IDD to the server 200. Upon reception of the device identification data IDD by the server 200, the execution device 210 in the server 200 stores the received device identification data IDD in the storage device 220. After Step S111, the execution device 210 in the server 200 terminates the current device registration control.

### (Normal Update Control)

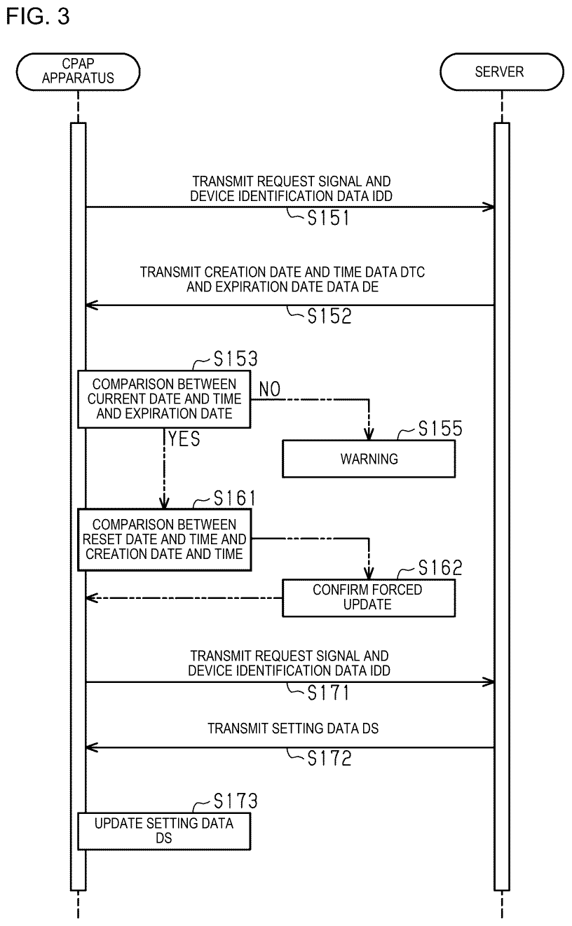

Normal update control performed by the CPAP apparatus 100 and the server 200 will now be described with reference to FIG. 3. The normal update control is the control to update the device-side setting data DS1. The normal update control may be performed in parallel between each of the multiple CPAP apparatuses 100 and the server 200. In the first embodiment, for example, when the user, the patient who borrowed the CPAP apparatus 100 from the medical facility, performs an operation to request the normal update control using the operation switch 20, the execution device 51 in the CPAP apparatus 100 starts the normal update control. In other words, in response to the request for update of the device-side setting data DS1, the execution device 51 in the CPAP apparatus 100 starts the normal update control.

As illustrated in FIG. 3, at the start of the normal update control, the execution device 51 in the control unit 50 in the CPAP apparatus 100 performs Step S151. In Step S151, the execution device 51 in the CPAP apparatus 100 transmits a request signal to request the creation date and time data DTC and the expiration date data DE to the server 200. At this time, the execution device 51 in the CPAP apparatus 100 also transmits the device identification data IDD to the server 200. Upon reception of the request signal and so on by the server 200, the execution device 210 in the server 200 goes to Step S152.

In Step S152, the execution device 210 in the server 200 transmits the creation date and time data DTC and the expiration date data DE to the CPAP apparatus 100. At this time, the execution device 210 in the server 200 identifies the server-side setting data DS2 about the CPAP apparatus 100 associated with the device identification data IDD acquired in Step S151. The execution device 210 in the server 200 transmits the creation date and time data DTC associated with the identified server-side setting data DS2 and so on to the CPAP apparatus 100 associated with the device identification data IDD. Upon reception of the creation date and time data DTC and the expiration date data DE by the CPAP apparatus 100, the execution device 51 in the CPAP apparatus 100 goes to Step S153. In the first embodiment, Step S152 is a step to acquire the creation date and time data DTC indicating the creation date and time of the server-side setting data DS2 to be acquired in update in response to a request for the update of the device-side setting data DS1. In other words, Step S152 is an example of a creation timing acquiring process. In addition, Step S152 is an example of an expiration date acquiring process to acquire the expiration date data DE indicating the expiration date of the server-side setting data DS2 to be acquired in update in response to a request for the update of the device-side setting data DS1.

In Step S153, the execution device 51 in the CPAP apparatus 100 determines whether the current date and time is before the expiration date indicated by the expiration date data DE acquired in Step S152. The current date and time here is the date and time when Step S153 is performed. In other words, the current date and time here is the date and time when the update of the device-side setting data DS1 is requested.

If the execution device 51 determines that the current date and time is after the expiration date indicated by the expiration date data DE acquired in Step S152 (NO in Step S153), the execution device 51 goes to Step S155.

In Step S155, the execution device 51 in the CPAP apparatus 100 performs a warning process to issue a warning to the user of the CPAP apparatus 100. For example, the execution device 51 outputs a control signal to the display 30 to warn the user of the CPAP apparatus 100 that the current date and time passes the expiration date of the server-side setting data DS2 with the display 30. In addition, in Step S155, the execution device 51 in the CPAP apparatus 100 transmits update result data DR indicating that the device-side setting data DS1 stored in the storage device 52 is not updated to the server 200. In other words, Step S155 includes a result transmitting process to transmit the update result data DR indicating whether the device-side setting data DS1 stored in the storage device 52 is updated to an external device. In the first embodiment, the server 200 is an example of the external device. After Step S155, the execution device 51 terminates the current normal update control.

If the execution device 51 determines that the current date and time is before the expiration date indicated by the expiration date data DE acquired in Step S152 (YES in Step S153), the execution device 51 goes to Step S161.

In Step S161, the execution device 51 in the CPAP apparatus 100 determines whether the reset date and time indicated by the reset date and time data DTR stored in the storage device 52 is before the creation date and time indicated by the creation date and time data DTC acquired in Step S152. In the first embodiment, Step S153 and Step S161 are an example of a determination process to determine whether a specific condition is met. If the requirement in Step S153 is met and the requirement in Step S161 is met, the execution device 51 in the CPAP apparatus 100 determines that the specific condition is met. In other words, in the first embodiment, the specific condition includes a requirement in which the current date and time is before the expiration date indicated by the expiration date data DE acquired in Step S152 and the reset date and time indicated by the reset date and time data DTR stored in the storage device 52 is before the creation date and time indicated by the creation date and time data DTC acquired in Step S152. If the execution device 51 determines that the reset date and time indicated by the reset date and time data DTR is after the creation date and time indicated by the creation date and time data DTC (NO in Step S161), the execution device 51 goes to Step S162. In other words, if the execution device 51 in the CPAP apparatus 100 determines that the specific condition is not met in the determination process, the execution device 51 in the CPAP apparatus 100 goes to Step S162.

In Step S162, the execution device 51 in the CPAP apparatus 100 confirms whether forced update of the device-side setting data DS1 stored in the storage device 52 is required with the user of the CPAP apparatus 100. The execution device 51 performs, for example, the following process. First, the execution device 51 outputs a control signal to the display 30 to display options indicating whether the forced update of the device-side setting data DS1 is accepted in the display 30. Here, if the user of the CPAP apparatus 100 selects no option using the operation switch 20, the execution device 51 continues to display the options in the display 30. If the user of the CPAP apparatus 100 selects an option not to accept the forced update using the operation switch 20, the execution device 51 terminates the current normal update control. At this time, the execution device 51 in the CPAP apparatus 100 transmits the update result data DR indicating that the device-side setting data DS1 stored in the storage device 52 is not updated to the server 200. In other words, Step S162 includes the result transmitting process to transmit the update result data DR indicating whether the device-side setting data DS1 stored in the storage device 52 is updated to an external device. If the user of the CPAP apparatus 100 selects an option to accept the forced update using the operation switch 20, the execution device 51 goes to Step S171. In the first embodiment, Step S162 is an example of a confirmation process to confirm whether update of the device-side setting data DS1 is required with the user of the CPAP apparatus 100. Accordingly, if the execution device 51 accepts the request for update of the device-side setting data DS1 in the confirmation process, the execution device 51 goes to Step S171 regardless of the specific condition.

If the execution device 51 determines that the reset date and time indicated by the reset date and time data DTR is before the creation date and time indicated by the creation date and time data DTC (YES in Step S161), the execution device 51 goes to Step S171. In other words, if the execution device 51 in the CPAP apparatus 100 determines that the specific condition is met in the determination process, the execution device 51 in the CPAP apparatus 100 goes to Step S171.

In Step S171, the execution device 51 in the CPAP apparatus 100 transmits a request signal to request the server-side setting data DS2 associated with the creation date and time data DTC acquired in Step S152 to the server 200. At this time, the execution device 51 in the CPAP apparatus 100 also transmits the device identification data IDD to the server 200. Upon reception of the request signal and so on by the server 200, the execution device 210 in the server 200 goes to Step S172.

In Step S172, the execution device 210 in the server 200 transmits the server-side setting data DS2 to the CPAP apparatus 100. Specifically, the execution device 210 in the server 200 transmits the server-side setting data DS2 associated with the creation date and time data DTC to the CPAP apparatus 100 associated with the device identification data IDD acquired in Step S151. Upon reception of the server-side setting data DS2 by the CPAP apparatus 100, the execution device 51 in the CPAP apparatus 100 goes to Step S173.

In Step S173, the execution device 51 in the CPAP apparatus 100 updates the device-side setting data DS1 stored in the storage device 52 to the server-side setting data DS2 associated with the creation date and time data DTC. As described above, if the execution device 51 determines that the specific condition is met in the determination process in Step S153 and S161, the execution device 51 goes to Step S171. When the execution device 51 performs Step S171 to Step S173 in the above manner, Step S171 to Step S173 correspond to an update process. As described above, if the execution device 51 accepts the request for update of the device-side setting data DS1 in the confirmation process in Step S162, the execution device 51 goes to Step S171 regardless of the specific condition. When the execution device 51 performs Step S171 to Step S173 in the above manner, Step S171 to Step S173 correspond to a forced update process responding to the request from the user of the CPAP apparatus 100. The execution device 51 in the CPAP apparatus 100 transmits the update result data DR indicating that the device-side setting data DS1 stored in the storage device 52 is updated to the server 200. In other words, Step S173 includes the result transmitting process to transmit the update result data DR indicating whether the device-side setting data DS1 stored in the storage device 52 is updated to an external device. After Step S173, the execution device 51 terminates the current normal update control.

### (Forced Update Control)

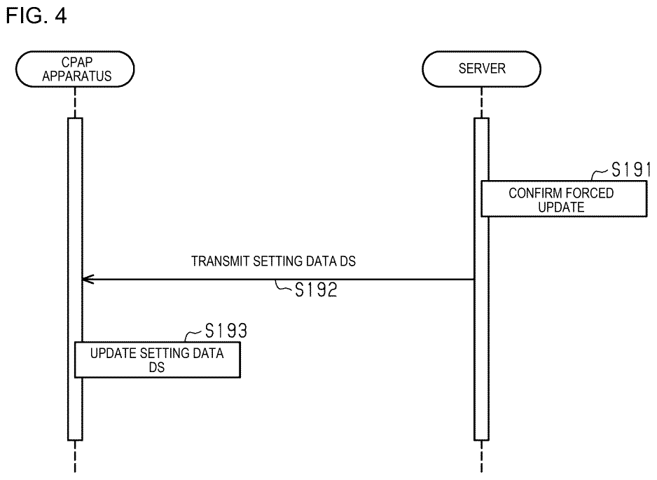

Forced update control performed by the CPAP apparatus 100 and the server 200 will now be described with reference to FIG. 4. The forced update control is the control to forcedly update the device-side setting data DS1 in response to a request from the server 200. The forced update control may be performed in parallel between each of the multiple CPAP apparatuses 100 and the server 200. In the first embodiment, if the server 200 receives the update result data DR indicating that the device-side setting data DS1 is not updated by the CPAP apparatus 100 in Step S155 or Step S162, the execution device 210 in the server 200 starts the forced update control. In other words, if the execution device 51 in the CPAP apparatus 100 transmits the update result data DR indicating that the device-side setting data DS1 is not updated in the result transmitting process, the execution device 210 in the server 200 starts the forced update control.

As illustrated in FIG. 4, at the start of the forced update control, the execution device 210 in the server 200 performs Step S191. In Step S191, the execution device 210 in the server 200 confirms whether the forced update of the device-side setting data DS1 stored in the storage device 52 in the CPAP apparatus 100 is required with the person in charge in the medical facility managing the server 200. Specifically, the execution device 210 in the server 200 performs, for example, the following process. First, the execution device 210 in the server 200 outputs a control signal to a display (not illustrated) connected to the server 200 to display options indicating whether the forced update of the device-side setting data DS1 is accepted in the display. Here, if the person in charge in the medical facility selects no option using the input device, the execution device 210 in the server 200 continues to display the options in the display. Examples of the input device are a keyboard and a pointing device, which are connected to the server 200. If the person in charge in the medical facility selects the option not to accept the forced update using the input device, the execution device 210 in the server 200 terminates the current forced update control. If the person in charge in the medical facility selects the option to accept the forced update using the input device, the execution device 210 in the server 200 goes to Step S192. In other words, upon acceptance of the request for update of the device-side setting data DS1 from the person in charge in the medical facility, the execution device 210 in the server 200 goes to Step S192 regardless of the specific condition.

In Step S192, the execution device 210 in the server 200 transmits the server-side setting data DS2 to the CPAP apparatus 100. Specifically, the execution device 210 in the server 200 transmits the server-side setting data DS2 associated with the creation date and time data DTC to the CPAP apparatus 100 associated with the device identification data IDD acquired in Step S151. Upon reception of the server-side setting data DS2 by the CPAP apparatus 100, the execution device 51 in the CPAP apparatus 100 goes to Step S193.

In Step S193, the execution device 51 in the CPAP apparatus 100 updates the device-side setting data DS1 stored in the storage device 52 to the server-side setting data DS2 associated with the creation date and time data DTC. In other words, if the execution device 51 in the CPAP apparatus 100 transmits the update result data DR indicating that the device-side setting data DS1 is not updated in the result transmitting process and accepts the request for update of the device-side setting data DS1 from an external device, the execution device 51 in the CPAP apparatus 100 updates the device-side setting data DS1 stored in the storage device 52 to the server-side setting data DS2 associated with the creation date and time data DTC regardless of the specific condition. In the first embodiment, Step S192 and Step S193 are an example of the forced update process responding to the request from an external device. The server 200 is an example of the external device. In addition, the execution device 51 in the CPAP apparatus 100 transmits the update result data DR indicating that the device-side setting data DS1 stored in the storage device 52 is updated to the server 200. After Step S193, the execution device 51 terminates the current forced update control.

### (Action in First Embodiment)

The CPAP apparatus 100 may be used, for example, in the following manner. First, it is assumed that the doctor in the medical facility diagnoses necessity of treatment using the CPAP apparatus 100 for a first patient. In this case, the first patient borrows the CPAP apparatus 100 from the medical facility to use the CPAP apparatus 100. Then, when the CPAP apparatus 100 is returned from the first patient to the medical facility, the person in charge in the medical facility performs a predetermined operation that is defined in advance for resetting the setting data DS using the operation switch 20 of the CPAP apparatus 100. The execution device 51 in the CPAP apparatus 100 performs the reset process to reset the setting data DS in response to the above operation, that is, in response to the reset request. In addition, the execution device 51 performs the storage process to store the reset timing at which the setting data DS has been reset in the reset process in the storage device 52 as the reset timing data. Then, it is assumed that the doctor in the medical facility diagnoses necessity of treatment using the CPAP apparatus 100 for a second patient different from the first patient. In this case, the second patient may borrow the CPAP apparatus 100 which the first patient used from the medical facility.

If the second patient performs the operation to request the normal update control using the operation switch 20 after borrowing the CPAP apparatus 100, the execution device 51 in the CPAP apparatus 100 starts the normal update control, as illustrated in FIG. 3. In Step S151 and Step S152, the execution device 51 in the CPAP apparatus 100 acquires the creation date and time data DTC indicating the creation date and time of the server-side setting data DS2 to be acquired in update in response to a request for the update of the device-side setting data DS1. In Step S153 and Step S161, the execution device 51 in the CPAP apparatus 100 performs the determination process to determine whether the specific condition is met. Here, the specific condition includes the requirement in which the reset date and time indicated by the reset date and time data DTR is before the creation date and time indicated by the creation date and time data DTC. If the execution device 51 in the CPAP apparatus 100 determines that the specific condition is met in the determination process, the execution device 51 in the CPAP apparatus 100 performs Step S171 to Step S173. In Step S171 to Step S173, the execution device 51 in the CPAP apparatus 100 performs the update process to update the device-side setting data DS1 stored in the storage device 52 to the server-side setting data DS2 associated with the creation date and time data DTC.

### (Effects of First Embodiment)

(1-1) For example, it is assumed that, when the second patient performs the operation to request the normal update control, that is, when update of the device-side setting data DS1 is requested from the second patient, the server-side setting data DS2 for the second patient has been created. In this case, the reset date and time indicated by the reset date and time data DTR is before the creation date and time indicated by the creation date and time data DTC. As a result, the execution device 51 in the CPAP apparatus 100 determines that the specific condition is met in the determination process and updates the device-side setting data DS1 stored in the storage device 52 to the server-side setting data DS2 associated with the creation date and time data DTC. Accordingly, if the creation timing of the server-side setting data DS2 is after the timing at which the CPAP apparatus 100 is returned from the first patient, that is, if it is determined that the server-side setting data DS2 is the data for the second patient, the setting data DS stored in the storage device 52 is updated.

In contrast, it is assumed that, when the second patient performs the operation to request the normal update control, that is, when update of the device-side setting data DS1 is requested from the second patient, the server-side setting data DS2 for the second patient has not been created. In this case, the reset date and time indicated by the reset date and time data DTR is after the creation date and time indicated by the creation date and time data DTC. Accordingly, the execution device 51 in the CPAP apparatus 100 determines that the specific condition is not met in the determination process. The execution device 51 in the CPAP apparatus 100 does not update the device-side setting data DS1 stored in the storage device 52 to the server-side setting data DS2 associated with the creation date and time data DTC. Consequently, if the creation timing of the server-side setting data DS2 is before the timing at which the CPAP apparatus 100 is returned from the first patient, that is, if it is not determined that the server-side setting data DS2 is the data for the second patient, the setting data DS stored in the storage device 52 is not updated. As a result, it is possible to suppress the update process to false setting data DS in the CPAP apparatus 100.

(1-2) As illustrated in FIG. 3, in Step S151 and Step S152, the execution device 51 in the CPAP apparatus 100 acquires the expiration date data DE indicating the expiration date of the server-side setting data DS2 to be acquired in update in response to a request for the update of the device-side setting data DS1. The specific condition in Step S153 and Step S161 includes the requirement in which the current date and time is before the expiration date indicated by the expiration date data DE and the reset date and time indicated by the reset date and time data DTR is before the creation date and time indicated by the creation date and time data DTC. With the above configuration, it is possible to suppress the update process to update the device-side setting data DS1 to the server-side setting data DS2 passing the expiration date.

(1-3) If the execution device 51 determines in Step S153 that the current date and time is after the expiration date indicated by the expiration date data DE, in Step S155, the execution device 51 in the CPAP apparatus 100 performs the warning process to issue a warning to the user of the CPAP apparatus 100. With the above configuration, the user of the CPAP apparatus 100 is capable of knowing that the expiration date of the server-side setting data DS2 is passed. If the user of the CPAP apparatus 100 is capable of knowing the situation in the above manner, the user is capable of recognizing that the user is required to visit the medical facility.

(1-4) If the execution device 51 determines in Step S161 that the reset date and time indicated by the reset date and time data DTR is after the creation date and time indicated by the creation date and time data DTC (NO in Step S161), the execution device 51 goes to Step S162. In other words, if the execution device 51 in the CPAP apparatus 100 determines that the specific condition is not met in the determination process, the execution device 51 in the CPAP apparatus 100 goes to Step S162. In Step S162, the execution device 51 in the CPAP apparatus 100 confirms whether the forced update of the device-side setting data DS1 stored in the storage device 52 is required with the user of the CPAP apparatus 100. Upon acceptance of the request for update of the device-side setting data DS1 in the confirmation process, the execution device 51 goes to Step S171 regardless of the specific condition. In Step S171 to Step S173, the execution device 51 in the CPAP apparatus 100 performs the forced update process to update the device-side setting data DS1 stored in the storage device 52 to the server-side setting data DS2 associated with the creation date and time data DTC. With the above configuration, even in a situation in which the specific condition is not met, it is possible to realize the forced update of the setting data DS in response to the request from the user of the CPAP apparatus 100.

(1-5) In Step S155, the execution device 51 in the CPAP apparatus 100 transmits the update result data DR indicating that the device-side setting data DS1 stored in the storage device 52 is not updated to the server 200. Similarly, if the device-side setting data DS1 is not updated, in Step S162, the execution device 51 in the CPAP apparatus 100 transmits the update result data DR indicating that the device-side setting data DS1 stored in the storage device 52 is not updated to the server 200. In Step S173, the execution device 51 in the CPAP apparatus 100 transmits the update result data DR indicating that the device-side setting data DS1 stored in the storage device 52 is updated to the server 200. In other words, Step S155, Step S162, and Step S173 include the result transmitting process to transmit the update result data DR indicating whether the device-side setting data DS1 stored in the storage device 52 is updated to an external device. With the above configuration, for example, the person in charge in the medical facility is capable of confirming the update result data DR acquired by the server 200 to know whether the setting data DS about the CPAP apparatus 100 is updated.

(1-6) If the server 200 receives the update result data DR indicating that the device-side setting data DS1 is not updated by the CPAP apparatus 100 in Step S155 or Step S162, the execution device 210 in the server 200 starts the forced update control, as illustrated in FIG. 4. In Step S191, the execution device 210 in the server 200 confirms whether the forced update of the device-side setting data DS1 stored in the storage device 52 in the CPAP apparatus 100 is required with the person in charge in the medical facility managing the server 200. Here, upon acceptance of the request for update of the device-side setting data DS1 from the person in charge in the medical facility, the execution device 210 in the server 200 goes to Step S192 regardless of the specific condition. In Step S192 and Step S193, the execution device 51 in the CPAP apparatus 100 updates the device-side setting data DS1 stored in the storage device 52 to the server-side setting data DS2 associated with the creation date and time data DTC. In other words, if the execution device 51 in the CPAP apparatus 100 transmits the update result data DR indicating that the device-side setting data DS1 is not updated in the result transmitting process and accepts the request for update of the device-side setting data DS1 from an external device, the execution device 51 in the CPAP apparatus 100 performs the forced update process to update the device-side setting data DS1 stored in the storage device 52 to the server-side setting data DS2 associated with the creation date and time data DTC regardless of the specific condition. With the above configuration, even in the situation in which the specific condition is not met, it is possible to realize the forced update of the setting data DS in response to the request from an external device.

### Second Embodiment

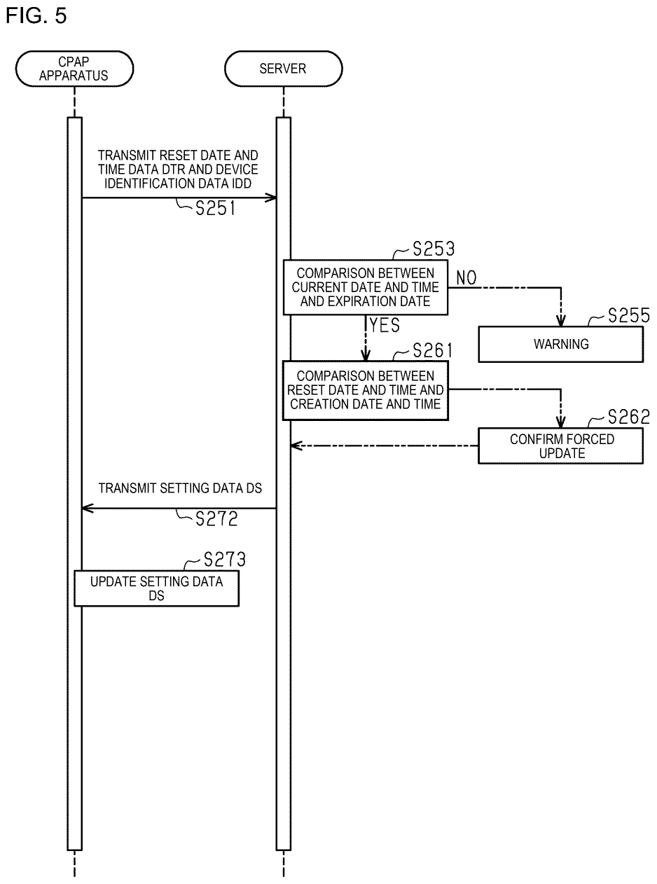

A second embodiment of the present disclosure will herein be described with reference to FIG. 5. The second embodiment differs from the first embodiment in that setting update control is performed, instead of the normal update control and the forced update control. In the second embodiment, the execution device 51 in the CPAP apparatus 100 corresponds to a device-side execution device. The storage device 52 in the CPAP apparatus 100 corresponds to a device-side storage device. The execution device 210 in the server 200 corresponds to a server-side execution device. The storage device 220 in the server 200 corresponds to a server-side storage device. The control program 221 stored in the storage device 220 in the server 200 is an example of an update program of the CPAP setting data. The execution device 210 in the server 200 executes the control program 221 to perform various processes concerning an update method of the CPAP setting data. The description of the second embodiment focuses on points different from the first embodiment. The same reference numerals and letters are used in the second embodiment to identify the same components as in the first embodiment and description of such components is omitted or simplified.

### (Setting Update Control)

The setting update control performed by the CPAP apparatus 100 and the server 200 will now be described with reference to FIG. 5. The setting update control is the control to update the device-side setting data DS1. The setting update control may be performed in parallel between each of the multiple CPAP apparatuses 100 and the server 200. In the second embodiment, for example, if the user, the patient who borrowed the CPAP apparatus 100 from the medical facility, performs an operation to request the setting update control using the operation switch 20, the execution device 51 in the CPAP apparatus 100 starts the setting update control. In other words, in response to the request for update of the device-side setting data DS1, the execution device 51 in the CPAP apparatus 100 starts the setting update control.

As illustrated in FIG. 5, at the start of the setting update control, the execution device 51 in the control unit 50 in the CPAP apparatus 100 performs Step S251. In Step S251, the execution device 51 in the CPAP apparatus 100 transmits the reset date and time data DTR and the device identification data IDD to the server 200. In the second embodiment, Step S251 is an example of a reset timing acquiring process to acquire the reset timing data in response to the request for update of the device-side setting data DS1. Upon reception of the reset date and time data DTR and so on by the server 200, the execution device 210 in the server 200 goes to Step S253.

In Step S253, the execution device 210 in the server 200 acquires the expiration date data DE stored in the storage device 220. This step is an example of the expiration date acquiring process to acquire the expiration date data DE indicating the expiration date of the server-side setting data DS2 to be transmitted in update in response to a request for update of the device-side setting data DS1. The execution device 210 in the server 200 determines whether the current date and time is before the expiration date indicated by the expiration date data DE. The current date and time here is the date and time when Step S253 is performed. In other words, the current date and time here is the date and time when the update of the device-side setting data DS1 is requested.

If the execution device 210 determines that the current date and time is after the expiration date indicated by the expiration date data DE (NO in Step S253), the execution device 210 goes to Step S255.

In Step S255, the execution device 210 in the server 200 performs the warning process to issue a warning to the user of the CPAP apparatus 100. For example, the execution device 210 in the server 200 outputs a control signal to the CPAP apparatus 100 to warn the user of the CPAP apparatus 100 that the current date and time passes the expiration date of the server-side setting data DS2 via the display 30 of the CPAP apparatus 100. In this case, the execution device 210 in the server 200 terminates the current setting update control.

If the execution device 210 determines that the current date and time is before the expiration date indicated by the expiration date data DE (YES in Step S253), the execution device 210 goes to Step S261.

In Step S261, the execution device 210 in the server 200 acquires the creation date and time data DTC stored in the storage device 220. This step is a step to acquire the creation date and time data DTC indicating the creation date and time of the server-side setting data DS2 in response to the request for update of the device-side setting data DS1. In other words, this step is an example of the creation timing acquiring process. The execution device 210 in the server 200 determines whether the reset date and time indicated by the reset date and time data DTR is before the creation date and time indicated by the creation date and time data DTC. In the second embodiment, Step S253 and Step S261 are an example of the determination process to determine whether the specific condition is met. If the requirement in Step S253 is met and the requirement in Step S261 is met, the execution device 210 in the server 200 determines that the specific condition is met. In other words, in the second embodiment, the specific condition includes the requirement in which the current date and time is before the expiration date indicated by the expiration date data DE and the reset date and time indicated by the reset date and time data DTR is before the creation date and time indicated by the creation date and time data DTC. If the execution device 210 determines that the reset date and time indicated by the reset date and time data DTR is after the creation date and time indicated by the creation date and time data DTC (NO in Step S261), the execution device 210 goes to Step S262. In other words, if the execution device 210 in the server 200 determines that the specific condition is not met in the determination process, the execution device 210 in the server 200 goes to Step S262.

In Step S262, the execution device 210 in the server 200 confirms whether the forced update of the device-side setting data DS1 stored in the storage device 52 in the CPAP apparatus 100 is required with the person in charge in the medical facility managing the server 200. Specifically, the execution device 210 in the server 200 performs, for example, the following process. First, the execution device 210 in the server 200 outputs a control signal to the display (not illustrated) connected to the server 200 to display options indicating whether the forced update of the device-side setting data DS1 is accepted in the display. Here, if the person in charge in the medical facility selects no option using the input device, the execution device 210 in the server 200 continues to display the options in the display. Examples of the input device are a keyboard and a pointing device, which are connected to the server 200. If the person in charge in the medical facility selects the option not to accept the forced update using the input device, the execution device 210 in the server 200 terminates the current setting update control. If the person in charge in the medical facility selects the option to accept the forced update using the input device, the execution device 210 in the server 200 goes to Step S272. In other words, upon acceptance of the request for update of the device-side setting data DS1 from the person in charge in the medical facility, the execution device 210 in the server 200 goes to Step S272 regardless of the specific condition.

If the execution device 210 determines that the reset date and time indicated by the reset date and time data DTR is before the creation date and time indicated by the creation date and time data DTC (YES in Step S261), the execution device 210 goes to Step S272. In other words, if the execution device 210 in the server 200 determines that the specific condition is met in the determination process, the execution device 210 in the server 200 goes to Step S272.

In Step S272, the execution device 210 in the server 200 transmits the server-side setting data DS2 to the CPAP apparatus 100. Specifically, the execution device 210 in the server 200 transmits the server-side setting data DS2 associated with the creation date and time data DTC to the CPAP apparatus 100 associated with the device identification data IDD acquired in Step S251. Upon reception of the server-side setting data DS2 by the CPAP apparatus 100, the execution device 51 in the CPAP apparatus 100 goes to Step S273.

In Step S273, the execution device 51 in the CPAP apparatus 100 updates the device-side setting data DS1 stored in the storage device 52 to the server-side setting data DS2 associated with the creation date and time data DTC. As described above, if the execution device 210 in the server 200 determines that the specific condition is met in the determination process in Step S253 and Step S261, the execution device 210 in the server 200 goes to Step S272. When Step S272 and Step S273 are performed in the above manner, Step S272 and Step S273 correspond to the update process. In other words, the execution device 210 in the server 200 transmits the server-side setting data DS2 associated with the creation date and time data DTC to the CPAP apparatus 100 to update the device-side setting data DS1 stored in the storage device 52 to the server-side setting data DS2 associated with the creation date and time data DTC. As described above, if the execution device 210 in the server 200 accepts the request for update of the device-side setting data DS1 in the confirmation process in Step S262, the execution device 210 in the server 200 goes to Step S272 regardless of the specific condition. When Step S272 and Step S273 are performed in the above manner, Step S272 and Step S273 correspond to the forced update process responding to the request from an external device. Here, the server 200 is an example of the external device. The execution device 51 in the CPAP apparatus 100 transmits the update result data DR indicating that the device-side setting data DS1 stored in the storage device 52 is updated to the server 200. In other words, Step S273 includes the result transmitting process to transmit the update result data DR indicating whether the device-side setting data DS1 stored in the storage device 52 is updated to an external device. After Step S273, the execution device 51 terminates the current setting update control.

### (Effects of Second Embodiment)

The second embodiment achieves the same effects as in (1-1) to (1-3), (1-5), and (1-6) described above.

### Third Embodiment

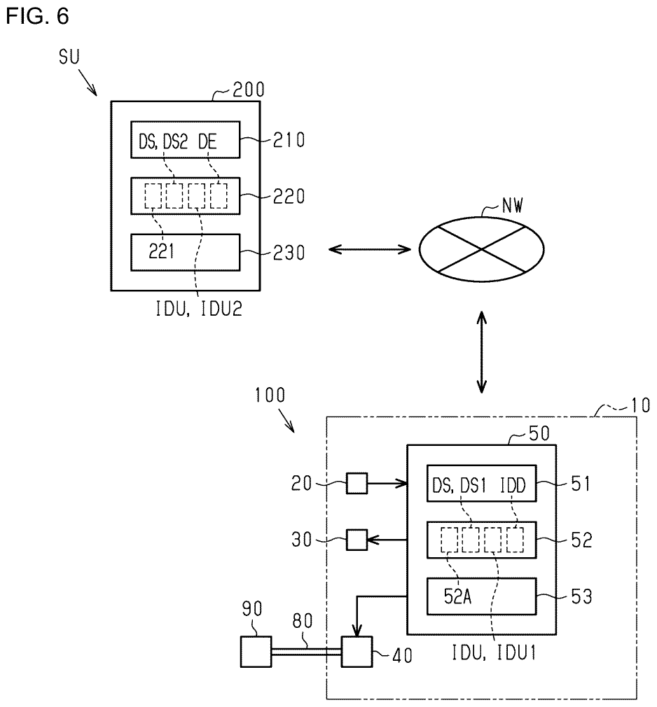

A third embodiment of the present disclosure will herein be described with reference to FIG. 6 to FIG. 9. The third embodiment differs from the first embodiment in the configuration of part of the CPAP apparatus 100 and the server 200. The third embodiment differs from the first embodiment in the configuration of the normal update control and the forced update control. The third embodiment differs from the first embodiment in that correlation control is performed. The description of the third embodiment focuses on points different from the first embodiment. The same reference numerals and letters are used in the third embodiment to identify the same components as in the first embodiment and description of such components is omitted or simplified.

### (Schematic Configuration of Update System)

As illustrated in FIG. 6, the storage device 52 in the CPAP apparatus 100 is capable of storing user identification data IDU, instead of the reset date and time data DTR. The user identification data IDU is data indicating information for identifying the user of the CPAP apparatus 100, that is, the patient using the CPAP apparatus 100. An example of the user identification data IDU is a number of a dozen digits. The execution device 51 stores the user identification data IDU in the storage device 52 each time the correlation control described below is performed. In other words, the user identification data IDU is writable data. The user identification data IDU stored in the storage device 52 in the CPAP apparatus 100 may be hereinafter referred to as device-side user identification data IDU1. The device-side user identification data IDU1 is an example of first identification data indicating the user.

The storage device 220 in the server 200 is capable of storing the user identification data IDU, instead of the creation date and time data DTC. The user identification data IDU stored in the storage device 220 in the server 200 may be hereinafter referred to as server-side user identification data IDU2. The server-side user identification data IDU2 is an example of second identification data indicating the user.

In the third embodiment, the server-side user identification data IDU2 is associated with the server-side setting data DS2 in the following manner. As described above, the patient visits the medical facility for use of the CPAP apparatus 100. At this time, the person in charge in the medical facility creates the user identification data IDU for the patient. In response to a predetermined operation by the person in charge in the medical facility using the input device connected to the server 200, the execution device 210 in the server 200 stores the created user identification data IDU in the storage device 220 as the server-side user identification data IDU2. After the patient visit, the person in charge in the medical facility creates the setting data DS based on the content of diagnosis for the patient. In response to a predetermined operation by the person in charge in the medical facility using the input device connected to the server 200, the execution device 210 in the server 200 stores the created setting data DS in the storage device 220 as the server-side setting data DS2. At this time, the execution device 210 in the server 200 associates the server-side setting data DS2 with the server-side user identification data IDU2 to store the server-side setting data DS2 and the server-side user identification data IDU2, which are associated with each other, in the storage device 220.

### (Correlation Control)

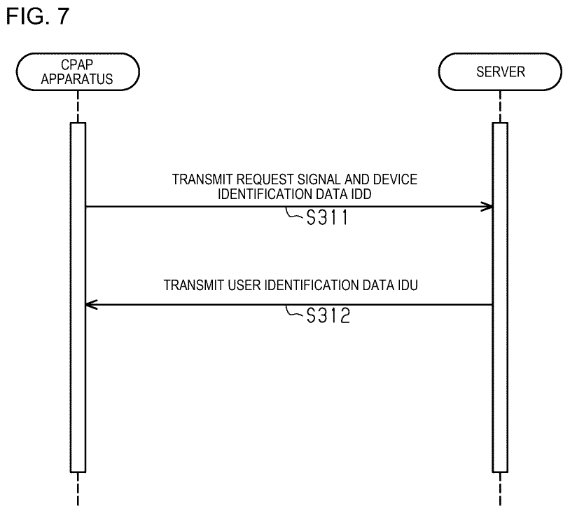

The correlation control performed by the CPAP apparatus 100 and the server 200 will now be described with reference to FIG. 7. The correlation control is the control to correlate the user identification data IDU in the CPAP apparatus 100 with the user identification data IDU in the server 200. The correlation control may be performed in parallel between each of the multiple CPAP apparatuses 100 and the server 200. In the third embodiment, for example, when the medical facility lends the CPAP apparatus 100 to the patient, in response to a predetermined operation by the person in charge in the medical facility using the operation switch 20 of the CPAP apparatus 100, the execution device 51 in the CPAP apparatus 100 starts the correlation control. Accordingly, in the third embodiment, the correlation control is performed between the target CPAP apparatus 100 and the server 200 each time the medical facility lends the CPAP apparatus 100 to the patient.

As illustrated in FIG. 7, at the start of the correlation control, the execution device 51 in the control unit 50 in the CPAP apparatus 100 performs Step S311. In Step S311, the execution device 51 in the CPAP apparatus 100 transmits a request signal to request the user identification data IDU to the server 200. At this time, the execution device 51 in the CPAP apparatus 100 also transmits the device identification data IDD to the server 200. Upon reception of the request signal and so on by the server 200, the execution device 210 in the server 200 goes to Step S312.

In Step S312, the execution device 210 in the server 200 identifies the user identification data IDU about the patient using the CPAP apparatus 100. The execution device 210 in the server 200 associates the identified user identification data IDU with the device identification data IDD acquired in Step S311 to store the user identification data IDU and the device identification data IDD, which are associated with each other, in the storage device 220. The execution device 210 in the server 200 transmits the identified user identification data IDU to the CPAP apparatus 100 associated with the device identification data IDD acquired in Step S311. Upon reception of the user identification data IDU by the CPAP apparatus 100, the execution device 51 in the CPAP apparatus 100 stores the user identification data IDU in the storage device 52 as the device-side user identification data IDU1. When the storage device 52 has already stored the device-side user identification data IDU1 at the time of Step S312, the execution device 51 rewrites the device-side user identification data IDU1 stored in the storage device 52 to new device-side user identification data IDU1. In the third embodiment, Step S312 is an example of the storage process to store the first identification data indicating a rewritable user in the storage device 52. After Step S312, the execution device 51 in the CPAP apparatus 100 terminates the current correlation control.

### (Normal Update Control)

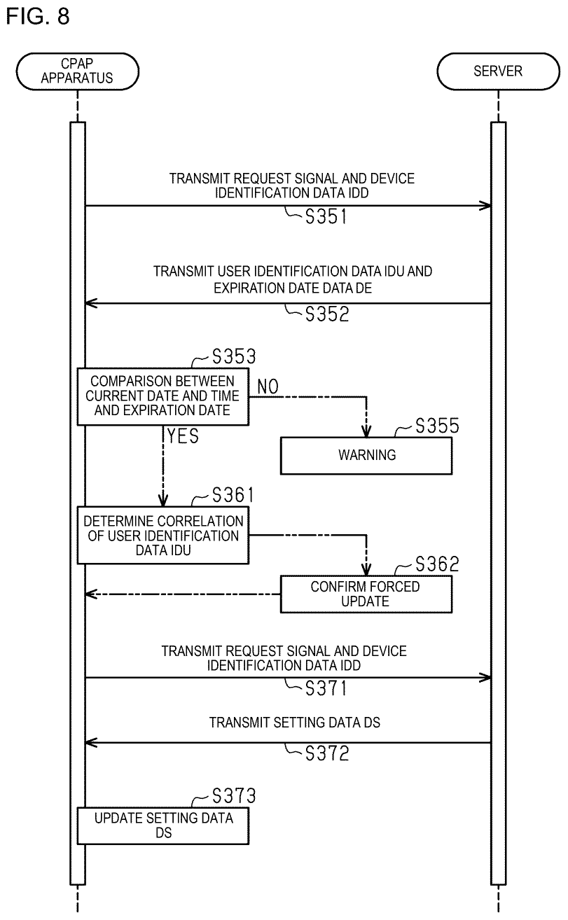

The normal update control performed by the CPAP apparatus 100 and the server 200 will now be described with reference to FIG. 8. The normal update control is the control to update the device-side setting data DS1. The normal update control may be performed in parallel between each of the multiple CPAP apparatuses 100 and the server 200. In the third embodiment, for example, when the user, the patient who borrowed the CPAP apparatus 100 from the medical facility, performs the operation to request the normal update control using the operation switch 20, the execution device 51 in the CPAP apparatus 100 starts the normal update control. In other words, in response to the request for update of the device-side setting data DS1, the execution device 51 in the CPAP apparatus 100 starts the normal update control.

As illustrated in FIG. 8, at the start of the normal update control, the execution device 51 in the control unit 50 in the CPAP apparatus 100 performs Step S351. In Step S351, the execution device 51 in the CPAP apparatus 100 transmits a request signal to request the server-side user identification data IDU2 and the expiration date data DE to the server 200. At this time, the execution device 51 in the CPAP apparatus 100 also transmits the device identification data IDD to the server 200. Upon reception of the request signal and so on by the server 200, the execution device 210 in the server 200 goes to Step S352.

In Step S352, the execution device 210 in the server 200 transmits the server-side user identification data IDU2 and the expiration date data DE to the CPAP apparatus 100. Specifically, the execution device 210 in the server 200 identifies the server-side setting data DS2 about the CPAP apparatus 100 associated with the device identification data IDD acquired in Step S351. The execution device 210 in the server 200 transmits the server-side user identification data IDU2 associated with the identified server-side setting data DS2 and the expiration date data DE to the CPAP apparatus 100 associated with the device identification data IDD acquired in Step S351. Upon reception of the server-side user identification data IDU2 and the expiration date data DE by the CPAP apparatus 100, the execution device 51 in the CPAP apparatus 100 goes to Step S353. In the third embodiment, Step S352 is a step to acquire the server-side user identification data IDU2 associated with the server-side setting data DS2 to be acquired in update in response to a request for the update of the device-side setting data DS1. In other words, Step S352 is an example of an identification data acquiring process. In addition, Step S352 is an example of the expiration date acquiring process to acquire the expiration date data DE indicating the expiration date of the server-side setting data DS2 to be acquired in update in response to a request for the update of the device-side setting data DS1.

In Step S353, the execution device 51 in the CPAP apparatus 100 determines whether the current date and time is before the expiration date indicated by the expiration date data DE acquired in Step S352. The current date and time here is the date and time when Step S353 is performed. In other words, the current date and time here is the date and time when the update of the device-side setting data DS1 is requested.

If the execution device 51 determines that the current date and time is after the expiration date indicated by the expiration date data DE acquired in Step S352 (NO in Step S353), the execution device 51 goes to Step S355.

In Step S355, the execution device 51 in the CPAP apparatus 100 performs the warning process to issue a warning to the user of the CPAP apparatus 100. For example, the execution device 51 outputs a control signal to the display 30 to warn the user of the CPAP apparatus 100 that the current date and time passes the expiration date of the server-side setting data DS2 with the display 30. In addition, in Step S355, the execution device 51 in the CPAP apparatus 100 transmits the update result data DR indicating that the device-side setting data DS1 stored in the storage device 52 is not updated to the server 200. In other words, Step S355 includes the result transmitting process to transmit the update result data DR indicating whether the device-side setting data DS1 stored in the storage device 52 is updated to an external device. In the third embodiment, the server 200 is an example of the external device. After Step S355, the execution device 51 terminates the current normal update control.

If the execution device 51 determines that the current date and time is before the expiration date indicated by the expiration date data DE acquired in Step S352 (YES in Step S353), the execution device 51 goes to Step S361.

In Step S361, the execution device 51 in the CPAP apparatus 100 determines whether the device-side user identification data IDU1 is correlated with the server-side user identification data IDU2 in advance. In the third embodiment, the execution device 51 determines that the device-side user identification data IDU1 is correlated with the server-side user identification data IDU2 in advance if the device-side user identification data IDU1 is the same as the server-side user identification data IDU2. The execution device 51 determines that the device-side user identification data IDU1 is not correlated with the server-side user identification data IDU2 in advance if the device-side user identification data IDU1 is not the same as the server-side user identification data IDU2. In the third embodiment, Step S353 and Step S361 are an example of the determination process to determine whether the specific condition is met. The execution device 51 in the CPAP apparatus 100 determines that the specific condition is met if the requirement in Step S353 is met and the requirement in Step S361 is met. In other words, in the third embodiment, the specific condition includes the requirement in which the current date and time is before the expiration date indicated by the expiration date data DE acquired in Step S352 and the device-side user identification data IDU1 is correlated with the server-side user identification data IDU2 in advance. If the execution device 51 determines that the device-side user identification data IDU1 is not correlated with the server-side user identification data IDU2 in advance (NO in Step S361), the execution device 51 goes to Step S362. In other words, if the execution device 51 in the CPAP apparatus 100 determines that the specific condition is not met in the determination process, the execution device 51 in the CPAP apparatus 100 goes to Step S362.

In Step S362, the execution device 51 in the CPAP apparatus 100 confirms whether the forced update of the device-side setting data DS1 stored in the storage device 52 is required with the user of the CPAP apparatus 100. The execution device 51 performs, for example, the following process. First, the execution device 51 outputs a control signal to the display 30 to display options indicating whether the forced update of the device-side setting data DS1 is accepted in the display 30. Here, if the user of the CPAP apparatus 100 selects no option using the operation switch 20, the execution device 51 continues to display the options in the display 30. If the user of the CPAP apparatus 100 selects the option not to accept the forced update using the operation switch 20, the execution device 51 terminates the current normal update control. At this time, the execution device 51 in the CPAP apparatus 100 transmits the update result data DR indicating that the device-side setting data DS1 stored in the storage device 52 is not updated to the server 200. In other words, Step S362 includes the result transmitting process to transmit the update result data DR indicating whether the device-side setting data DS1 stored in the storage device 52 is updated to an external device. If the user of the CPAP apparatus 100 selects the option to accept the forced update using the operation switch 20, the execution device 51 goes to Step S371. In the third embodiment, Step S362 is an example of the confirmation process to confirm whether update of the device-side setting data DS1 is required with the user of the CPAP apparatus 100. Accordingly, if the execution device 51 accepts the request for update of the device-side setting data DS1 in the confirmation process, the execution device 51 goes to Step S371 regardless of the specific condition.

If the execution device 51 determines that the device-side user identification data IDU1 is correlated with the server-side user identification data IDU2 in advance (YES in Step S361), the execution device 51 goes to Step S371. In other words, if the execution device 51 in the CPAP apparatus 100 determines that the specific condition is met in the determination process, the execution device 51 in the CPAP apparatus 100 goes to Step S371.

In Step S371, the execution device 51 in the CPAP apparatus 100 transmits a request signal to request the server-side setting data DS2 associated with the server-side user identification data IDU2 acquired in Step S352 to the server 200. At this time, the execution device 51 in the CPAP apparatus 100 also transmits the device identification data IDD to the server 200. Upon reception of the request signal and so on by the server 200, the execution device 210 in the server 200 goes to Step S372.

In Step S372, the execution device 210 in the server 200 transmits the server-side setting data DS2 to the CPAP apparatus 100. Specifically, the execution device 210 in the server 200 transmits the server-side setting data DS2 associated with the server-side user identification data IDU2 to the CPAP apparatus 100 associated with the device identification data IDD acquired in Step S371. Upon reception of the server-side setting data DS2 by the CPAP apparatus 100, the execution device 51 in the CPAP apparatus 100 goes to Step S373.

In Step S373, the execution device 51 in the CPAP apparatus 100 updates the device-side setting data DS1 stored in the storage device 52 to the server-side setting data DS2 associated with the server-side user identification data IDU2. As described above, if the execution device 51 determines that the specific condition is met in the determination process in Step S353 and S361, the execution device 51 goes to Step S371. When the execution device 51 performs Step S371 to Step S373 in the above manner, Step S371 to Step S373 correspond to the update process. As described above, if the execution device 51 accepts the request for update of the device-side setting data DS1 in the confirmation process in Step S362, the execution device 51 goes to Step S371 regardless of the specific condition. When the execution device 51 performs Step S371 to Step S373 in the above manner, Step S371 to Step S373 correspond to the forced update process responding to the request from the user of the CPAP apparatus 100. The execution device 51 in the CPAP apparatus 100 transmits the update result data DR indicating that the device-side setting data DS1 stored in the storage device 52 is updated to the server 200. In other words, Step S373 includes the result transmitting process to transmit the update result data DR indicating whether the device-side setting data DS1 stored in the storage device 52 is updated to an external device. After Step S373, the execution device 51 terminates the current normal update control.

### (Forced Update Control)

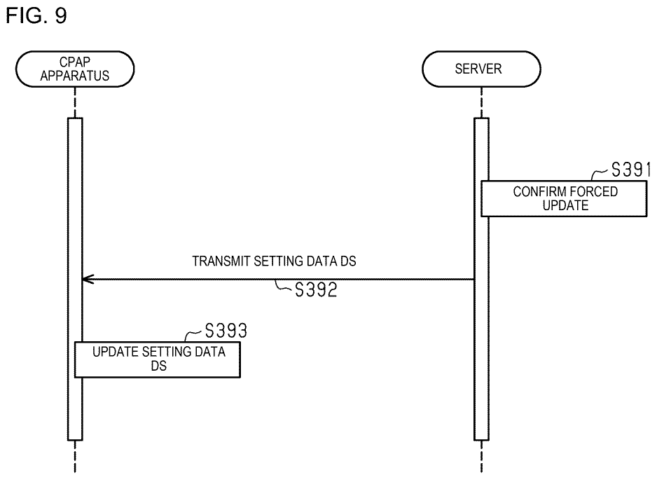

The forced update control performed by the CPAP apparatus 100 and the server 200 will now be described with reference to FIG. 9. The forced update control is the control to forcedly update the device-side setting data DS1 in response to a request from the server 200. The forced update control may be performed in parallel between each of the multiple CPAP apparatuses 100 and the server 200. In the third embodiment, if the server 200 receives the update result data DR indicating that the device-side setting data DS1 is not updated by the CPAP apparatus 100 in Step S355 or Step S362, the execution device 210 in the server 200 starts the forced update control. In other words, if the execution device 51 in the CPAP apparatus 100 transmits the update result data DR indicating that the device-side setting data DS1 is not updated in the result transmitting process, the execution device 210 in the server 200 starts the forced update control.

As illustrated in FIG. 9, at the start of the forced update control, the execution device 210 in the server 200 performs Step S391. In Step S391, the execution device 210 in the server 200 confirms whether the forced update of the device-side setting data DS1 stored in the storage device 52 in the CPAP apparatus 100 is required with the person in charge in the medical facility managing the server 200. Specifically, the execution device 210 in the server 200 performs, for example, the following process. First, the execution device 210 in the server 200 outputs a control signal to the display (not illustrated) connected to the server 200 to display options indicating whether the forced update of the device-side setting data DS1 is accepted in the display. Here, if the person in charge in the medical facility selects no option using the input device, the execution device 210 in the server 200 continues to display the options in the display. Examples of the input device are a keyboard and a pointing device, which are connected to the server 200. If the person in charge in the medical facility selects the option not to accept the forced update using the input device, the execution device 210 in the server 200 terminates the current forced update control. If the person in charge in the medical facility selects the option to accept the forced update using the input device, the execution device 210 in the server 200 goes to Step S392. In other words, upon acceptance of the request for update of the device-side setting data DS1 from the person in charge in the medical facility, the execution device 210 in the server 200 goes to Step S392 regardless of the specific condition.

In Step S392, the execution device 210 in the server 200 transmits the server-side setting data DS2 to the CPAP apparatus 100. Specifically, the execution device 210 in the server 200 transmits the server-side setting data DS2 associated with the server-side user identification data IDU2 to the CPAP apparatus 100 associated with the device identification data IDD acquired in Step S351. Upon reception of the server-side setting data DS2 by the CPAP apparatus 100, the execution device 51 in the CPAP apparatus 100 goes to Step S393.

In Step S393, the execution device 51 in the CPAP apparatus 100 updates the device-side setting data DS1 stored in the storage device 52 to the server-side setting data DS2 associated with the server-side user identification data IDU2. In other words, if the execution device 51 in the CPAP apparatus 100 transmits the update result data DR indicating that the device-side setting data DS1 is not updated in the result transmitting process and accepts the request for update of the device-side setting data DS1 from an external device, the execution device 51 in the CPAP apparatus 100 updates the device-side setting data DS1 stored in the storage device 52 to the server-side setting data DS2 associated with the server-side user identification data IDU2 regardless of the specific condition. In the third embodiment, Step S392 and Step S393 are an example of the forced update process responding to the request from an external device. The server 200 is an example of the external device. In addition, the execution device 51 in the CPAP apparatus 100 transmits the update result data DR indicating that the device-side setting data DS1 stored in the storage device 52 is updated to the server 200. After Step S393, the execution device 51 terminates the current forced update control.

### (Action in Third Embodiment)

The CPAP apparatus 100 may be used, for example, in the following manner. First, it is assumed that the doctor in the medical facility diagnoses necessity of treatment using the CPAP apparatus 100 for a first patient. In this case, the first patient borrows the CPAP apparatus 100 from the medical facility to use the CPAP apparatus 100. Then, after the CPAP apparatus 100 is returned from the first patient to the medical facility, it is assumed that the doctor in the medical facility diagnoses necessity of treatment using the CPAP apparatus 100 for a second patient different from the first patient. In this case, the second patient may borrow the CPAP apparatus 100 which the first patient used from the medical facility.

In the lending of the CPAP apparatus 100 from the medical facility to the second patient, if the person in charge in the medical facility performs an operation to request the correlation control using the operation switch 20 of the CPAP apparatus 100, the execution device 51 in the CPAP apparatus 100 starts the correlation control, as illustrated in FIG. 7. In Step S311 and Step S312, the execution device 51 in the CPAP apparatus 100 stores the acquired user identification data IDU in the storage device 52 as the device-side user identification data IDU1.

If the second patient performs the operation to request the normal update control using the operation switch 20 after borrowing the CPAP apparatus 100, the execution device 51 in the CPAP apparatus 100 starts the normal update control, as illustrated in FIG. 8. In Step S351 and Step S352, the execution device 51 in the CPAP apparatus 100 acquires the server-side user identification data IDU2 associated with the server-side setting data DS2 to be acquired in update in response to a request for the update of the device-side setting data DS1. In Step S353 and Step S361, the execution device 51 in the CPAP apparatus 100 performs the determination process to determine whether the specific condition is met. Here, the specific condition includes the requirement in which the device-side user identification data IDU1 is correlated with the server-side user identification data IDU2 in advance. If the execution device 51 in the CPAP apparatus 100 determines that the specific condition is met in the determination process, the execution device 51 in the CPAP apparatus 100 performs Step S371 to Step S373. In Step S371 to Step S373, the execution device 51 in the CPAP apparatus 100 performs the update process to update the device-side setting data DS1 stored in the storage device 52 to the server-side setting data DS2 associated with the server-side user identification data IDU2.

### (Effects of Third Embodiment)

The third embodiment achieves the effects of (3-1) described below, in addition to the same effects as in (1-2) to (1-6) described above.

(3-1) For example, it is assumed that, when the second patient performs the operation to request the normal update control, that is, when update of the device-side setting data DS1 is requested from the second patient, the server-side setting data DS2 for the second patient is created. In this case, both the device-side user identification data IDU1 and the server-side user identification data IDU2 indicate the second patient. As a result, the execution device 51 in the CPAP apparatus 100 determines that the specific condition is met in the determination process and updates the device-side setting data DS1 stored in the storage device 52 to the server-side setting data DS2 associated with the server-side user identification data IDU2. Accordingly, if the server-side setting data DS2 is determined to be the data for the second patient, the setting data DS stored in the storage device 52 is updated.

In contrast, for example, it is assumed that, when the second patient performs the operation to request the normal update control, that is, when update of the device-side setting data DS1 is requested from the second patient, the server-side setting data DS2 for the second patient is not created. In this case, the device-side user identification data IDU1 indicates the second patient while the server-side user identification data IDU2 indicates the first patient. Accordingly, the execution device 51 in the CPAP apparatus 100 determines that the specific condition is not met in the determination process. The execution device 51 in the CPAP apparatus 100 does not update the device-side setting data DS1 stored in the storage device 52 to the server-side setting data DS2 associated with the server-side user identification data IDU2. Accordingly, if the server-side setting data DS2 is not determined to be the data for the second patient, the setting data DS stored in the storage device 52 is not updated. This suppresses update to false setting data DS in the CPAP apparatus 100.

### Fourth Embodiment

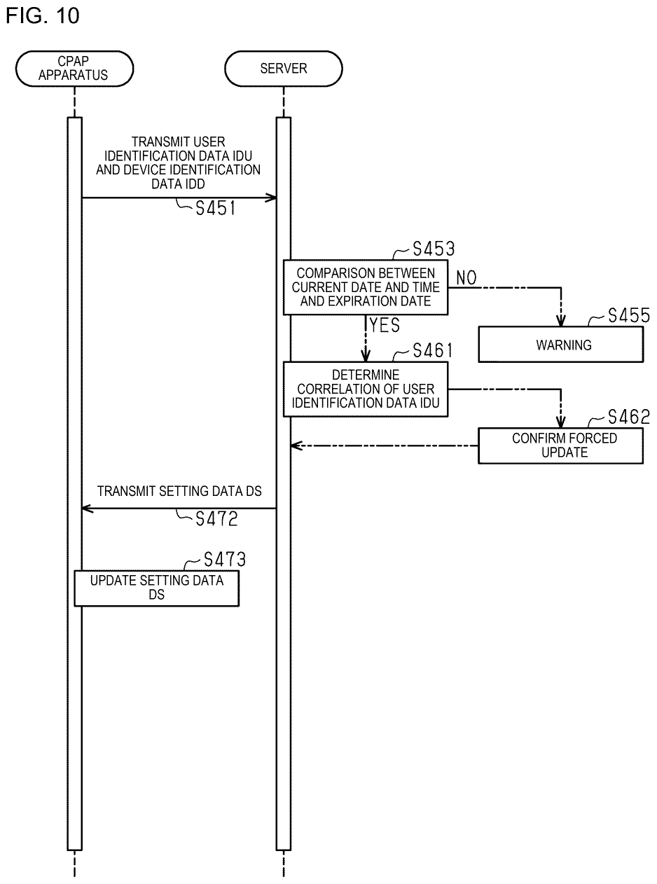

A fourth embodiment of the present disclosure will herein be described with reference to FIG. 10. The fourth embodiment differs from the third embodiment in that the setting update control is performed, instead of the normal update control and the forced update control. In the fourth embodiment, the execution device 51 in the CPAP apparatus 100 corresponds to the device-side execution device. The storage device 52 in the CPAP apparatus 100 corresponds to the device-side storage device. The execution device 210 in the server 200 corresponds to the server-side execution device. The storage device 220 in the server 200 corresponds to the server-side storage device. The control program 221 stored in the storage device 220 in the server 200 is an example of an update program of the CPAP setting data. The execution device 210 in the server 200 executes the control program 221 to perform various processes concerning an update method of the CPAP setting data. The description of the fourth embodiment focuses on points different from the third embodiment. The same reference numerals and letters are used in the fourth embodiment to identify the same components as in the third embodiment and description of such components is omitted or simplified.

### (Setting Update Control)

The setting update control performed by the CPAP apparatus 100 and the server 200 will now be described with reference to FIG. 10. The setting update control is the control to update the device-side setting data DS1. The setting update control may be performed in parallel between each of the multiple CPAP apparatuses 100 and the server 200. In the fourth embodiment, for example, if the user, the patient who borrowed the CPAP apparatus 100 from the medical facility, performs the operation to request the setting update control using the operation switch 20, the execution device 51 in the CPAP apparatus 100 starts the setting update control. In other words, in response to the request for update of the device-side setting data DS1, the execution device 51 in the CPAP apparatus 100 starts the setting update control.

As illustrated in FIG. 10, at the start of the setting update control, the execution device 51 in the control unit 50 in the CPAP apparatus 100 performs Step S451. In Step S451, the execution device 51 in the CPAP apparatus 100 transmits the device-side user identification data IDU1 and the device identification data IDD to the server 200. In the fourth embodiment, Step S451 is an example of a first identification data acquiring process to acquire the device-side user identification data IDU1 in response to the request for update of the device-side setting data DS1. Upon reception of the device-side user identification data IDU1 and so on by the server 200, the execution device 210 in the server 200 goes to Step S453.

In Step S453, the execution device 210 in the server 200 acquires the expiration date data DE stored in the storage device 220. This step is an example of the expiration date acquiring process to acquire the expiration date data DE indicating the expiration date of the server-side setting data DS2 to be transmitted in update in response to a request for update of the device-side setting data DS1. The execution device 210 in the server 200 determines whether the current date and time is before the expiration date indicated by the expiration date data DE. The current date and time here is the date and time when Step S453 is performed. In other words, the current date and time here is the date and time when the update of the device-side setting data DS1 is requested.

If the execution device 210 determines that the current date and time is after the expiration date indicated by the expiration date data DE (NO in Step S453), the execution device 210 goes to Step S455.

In Step S455, the execution device 210 in the server 200 performs the warning process to issue a warning to the user of the CPAP apparatus 100. For example, the execution device 210 in the server 200 outputs a control signal to the CPAP apparatus 100 to warn the user of the CPAP apparatus 100 that the current date and time passes the expiration date of the server-side setting data DS2 via the display 30 of the CPAP apparatus 100. In this case, the execution device 210 in the server 200 terminates the current setting update control.

If the execution device 210 determines that the current date and time is before the expiration date indicated by the expiration date data DE (YES in Step S453), the execution device 210 goes to Step S461.

In Step S461, the execution device 210 in the server 200 identifies the server-side setting data DS2 about the CPAP apparatus 100 associated with the device identification data IDD acquired in Step S451. The execution device 210 in the server 200 acquires the server-side user identification data IDU2 associated with the identified server-side setting data DS2 from the storage device 220. This step is an example of a second identification data acquiring process to acquire the server-side user identification data IDU2 associated with the server-side setting data DS2 in response to the request for update of the device-side setting data DS1. The execution device 210 in the server 200 determines whether the device-side user identification data IDU1 is correlated with the server-side user identification data IDU2 in advance. In the fourth embodiment, the execution device 210 determines that the device-side user identification data IDU1 is correlated with the server-side user identification data IDU2 in advance if the device-side user identification data IDU1 is the same as the server-side user identification data IDU2. The execution device 210 determines that the device-side user identification data IDU1 is not correlated with the server-side user identification data IDU2 in advance if the device-side user identification data IDU1 is not the same as the server-side user identification data IDU2. In the fourth embodiment, Step S453 and Step S461 are an example of the determination process to determine whether the specific condition is met. The execution device 210 in the server 200 determines that the specific condition is met if the requirement in Step S453 is met and the requirement in Step S461 is met. In other words, in the fourth embodiment, the specific condition includes the requirement in which the current date and time is before the expiration date indicated by the expiration date data DE and the device-side user identification data IDU1 is correlated with the server-side user identification data IDU2 in advance. If the execution device 210 determines that the device-side user identification data IDU1 is not correlated with the server-side user identification data IDU2 in advance (NO in Step S461), the execution device 210 goes to Step S462. In other words, if the execution device 210 in the server 200 determines that the specific condition is not met in the determination process, the execution device 210 in the server 200 goes to Step S462.

In Step S462, the execution device 210 in the server 200 confirms whether the forced update of the device-side setting data DS1 stored in the storage device 52 in the CPAP apparatus 100 is required with the person in charge in the medical facility managing the server 200. Specifically, the execution device 210 in the server 200 performs, for example, the following process. First, the execution device 210 in the server 200 outputs a control signal to the display (not illustrated) connected to the server 200 to display options indicating whether the forced update of the device-side setting data DS1 is accepted in the display. Here, if the person in charge in the medical facility selects no option using the input device, the execution device 210 in the server 200 continues to display the options in the display. Examples of the input device are a keyboard and a pointing device, which are connected to the server 200. If the person in charge in the medical facility selects the option not to accept the forced update using the input device, the execution device 210 in the server 200 terminates the current setting update control. If the person in charge in the medical facility selects the option to accept the forced update using the input device, the execution device 210 in the server 200 goes to Step S472. In other words, upon acceptance of the request for update of the device-side setting data DS1 from the person in charge in the medical facility, the execution device 210 in the server 200 goes to Step S472 regardless of the specific condition.

If the execution device 210 determines that device-side user identification data IDU1 is correlated with the server-side user identification data IDU2 in advance (YES in Step S461), the execution device 210 goes to Step S472. In other words, if the execution device 210 in the server 200 determines that the specific condition is met in the determination process, the execution device 210 in the server 200 goes to Step S472.

In Step S472, the execution device 210 in the server 200 transmits the server-side setting data DS2 to the CPAP apparatus 100. Specifically, the execution device 210 in the server 200 transmits the server-side setting data DS2 associated with the server-side user identification data IDU2 to the CPAP apparatus 100 associated with the device identification data IDD acquired in Step S451. Upon reception of the server-side setting data DS2 by the CPAP apparatus 100, the execution device 51 in the CPAP apparatus 100 goes to Step S473.

In Step S473, the execution device 51 in the CPAP apparatus 100 updates the device-side setting data DS1 stored in the storage device 52 to the server-side setting data DS2 associated with the server-side user identification data IDU2. As described above, if the execution device 210 in the server 200 determines that the specific condition is met in the determination process in Step S453 and Step S461, the execution device 210 in the server 200 goes to Step S472. When Step S472 and Step S473 are performed in the above manner, Step S472 and Step S473 correspond to the update process. In other words, the execution device 210 in the server 200 transmits the server-side setting data DS2 associated with the server-side user identification data IDU2 to the CPAP apparatus 100 to update the device-side setting data DS1 stored in the storage device 52 to the server-side setting data DS2 associated with the server-side user identification data IDU2. As described above, if the execution device 210 in the server 200 accepts the request for update of the device-side setting data DS1 in the confirmation process in Step S462, the execution device 210 in the server 200 goes to Step S472 regardless of the specific condition. When Step S472 and Step S473 are performed in the above manner, Step S472 and Step S473 correspond to the forced update process responding to the request from an external device. Here, the server 200 is an example of the external device. The execution device 51 in the CPAP apparatus 100 transmits the update result data DR indicating that the device-side setting data DS1 stored in the storage device 52 is updated to the server 200. In other words, Step S473 includes the result transmitting process to transmit the update result data DR indicating whether the device-side setting data DS1 stored in the storage device 52 is updated to an external device. After Step S473, the execution device 51 terminates the current setting update control.

### (Effects of Fourth Embodiment)

The fourth embodiment achieves the same effects as in (1-2), (1-3), (1-5), (1-6), and (3-1) described above.

### (Modifications)

The first embodiment to the fourth embodiment described above may be modified in the following manner. The first embodiment to the fourth embodiment described above and modifications described below may be combined within the scope of technical consistency.

In the first embodiment described above, the normal update control may be modified.

For example, in Step S155, the warning process is not necessarily performed. As a specific example, in Step S155, the execution device 51 in the CPAP apparatus 100 may confirm whether the process goes to Step S161 with the user of the CPAP apparatus 100. If the user of the CPAP apparatus 100 selects Step S161 using the operation switch 20, the execution device 51 goes to Step S161. The same applies to the second embodiment to the fourth embodiment.

For example, Step S155 may be omitted. As a specific example, if the execution device 51 determines in Step S153 that the current date and time is after the expiration date indicated by the expiration date data DE, the execution device 51 may terminate the current normal update control without performing the warning process in Step S155. The same applies to the second embodiment to the fourth embodiment.

For example, Step S153 may be omitted. As a specific example, the execution device 51 in the CPAP apparatus 100 may go to Step S161 after Step S152. In other words, the specific condition may be modified. The same applies to the second embodiment to the fourth embodiment.

For example, in Step S162, the execution device 51 in the CPAP apparatus 100 does not necessarily confirm whether update of the device-side setting data DS1 is required with the user of the CPAP apparatus 100. As a specific example, in Step S162, the execution device 51 in the CPAP apparatus 100 may perform the warning process to issue a warning to the user of the CPAP apparatus 100, as in Step S155. In this case, the execution device 51 may terminate the current normal update control after Step S162. The same applies to the third embodiment.

For example, Step S162 may be omitted. As a specific example, if the execution device 51 determines in Step S161 that the reset date and time indicated by the reset date and time data DTR is after the creation date and time indicated by the creation date and time data DTC, the execution device 51 may terminate the current normal update control without performing Step S162. The same applies to the third embodiment.

For example, Step S173 does not necessarily include the result transmitting process to transmit the update result data DR indicating whether the device-side setting data DS1 stored in the storage device 52 is updated to an external device. This applies to Step S155, Step S162, and Step S193. In addition, this applies to the second embodiment to the fourth embodiment.

In the first embodiment described above, the forced update control may be omitted. In this case, in the normal update control, the result transmitting process to transmit the update result data DR indicating whether the device-side setting data DS1 stored in the storage device 52 is updated to an external device is not necessarily performed. This applies to the third embodiment.

In the first embodiment described above, the configuration of the update system SU may be modified.

For example, the creation timing data indicating the creation timing of the server-side setting data DS2 may be modified. In other words, the creation timing data is not limited to the creation date and time data DTC. As a specific example, the creation timing data may be data indicating the elapsed period since a predetermined date and time that is defined in advance. Also in this case, the creation date and time of the server-side setting data DS2 can be identified based on the predetermined date and time that is defined in advance and the elapsed period indicated by the creation timing data. An example of the predetermined date and time that is defined in advance is the date and time when the server 200 is manufactured. This applies to the second embodiment.

For example, the data indicating the expiration date of the server-side setting data DS2 may be modified. As a specific example, the server-side setting data DS2 may be data indicating the elapsed period since a predetermined date and time that is defined in advance. An example of the predetermined date and time that is defined in advance is the creation date and time indicated by the creation date and time data DTC. This applies to the second embodiment.

For example, the main body 10 of the CPAP apparatus 100 does not necessarily include the operation switch 20. As a specific example, it is assumed that the control unit 50 in the CPAP apparatus 100 is capable of communicating with an input device outside the CPAP apparatus 100 via wireless communication or the like. With this configuration, the control unit 50 is capable of accepting an operation by the user of the CPAP apparatus 100 via the input device even without the operation switch 20. An example of the input device is a so-called smartphone. An example of the communication method of the wireless communication described above is Bluetooth (registered trademark).

For example, the main body 10 of the CPAP apparatus 100 does not necessarily include the display 30. As a specific example, it is assumed that the control unit 50 in the CPAP apparatus 100 is capable of communicating with a display device outside the CPAP apparatus 100 via the wireless communication or the like. With this configuration, the control unit 50 is capable of transmitting a variety of information to the user of the CPAP apparatus 100 via the display device even without the display 30. An example of the display device is a so-called smartphone. An example of the communication method of the wireless communication described above is wireless communication according to a Wi-Fi standard.

For example, the CPAP apparatus 100 is not necessarily provided with the hose 80 and the mask 90 only in terms of suppression of update to false setting data DS in the CPAP apparatus 100.

For example, the situation in which the CPAP apparatus 100 is used may be modified. As a specific example, the patient may buy the CPAP apparatus 100 from the medical facility to use the CPAP apparatus 100, in addition to borrowing the CPAP apparatus 100 from the medical facility by the patient. The technique in the present disclosure is applicable also in such a situation.

For example, the update system SU may include a portable storage medium, instead of the server 200. In other words, the execution device 51 in the CPAP apparatus 100 may acquire the setting data DS and so on stored in the portable storage medium in update of the device-side setting data DS1. In this configuration, it is sufficient for the portable storage medium to store the setting data DS, the creation date and time data DTC, and the expiration date data DE. An example of the portable storage medium is a so-called secure digital (SD) memory card. The SD memory card may be called an SD card or the like.

In the second embodiment described above, the setting update control may be modified.

For example, in Step S262, the execution device 210 in the server 200 does not necessarily confirm whether update of the device-side setting data DS1 is required with the person in charge in the medical facility managing the server 200. As a specific example, in Step S262, the execution device 210 in the server 200 may perform the warning process to issue a warning to the user of the CPAP apparatus 100, as in Step S255. In this case, the execution device 210 may terminate the current setting update control after Step S262. The same applies to the fourth embodiment.

For example, Step S262 may be omitted. As a specific example, if the execution device 210 determines in Step S261 that the reset date and time indicated by the reset date and time data DTR is after the creation date and time indicated by the creation date and time data DTC, the execution device 210 may terminate the current setting update control without performing Step S262. The same applies to the fourth embodiment.

In the third embodiment described above, the normal update control may be modified.

For example, the determination method in Step S361 may be modified. As a specific example, it is assumed that the symbol indicated by the device-side user identification data IDU1 for the second patient is “A123” and the symbol indicated by the server-side user identification data IDU2 for the second patient is “B123”. The device-side user identification data IDU1 may be correlated with the server-side user identification data IDU2 in advance in the state in which the symbol indicated by the device-side user identification data IDU1 is different from the symbol indicated by the server-side user identification data IDU2 in the above manner. In this case, in Step S361, if the symbol indicated by the device-side user identification data IDU1 is “A123” and the symbol indicated by the server-side user identification data IDU2 is “B123” even when the device-side user identification data IDU1 is not the same as the server-side user identification data IDU2, the execution device 51 in the CPAP apparatus 100 determines that the device-side user identification data IDU1 is correlated with the server-side user identification data IDU2 in advance.

In the third embodiment described above, the configuration of the update system SU may be modified.

For example, the configuration concerning the first identification data may be modified. As a specific example, the first identification data may be data specific to the patient or may be data that is created each time the patient visits the medical facility. In other words, the same first identification data may be continuously used for the same patient or the first identification data may be modified each time the patient visits the medical facility. The configuration of the first identification data indicating the user may be modified as long as the patient using the CPAP apparatus 100 can be identified using the first identification data. The same applies to the configuration concerning the second identification data. In addition, the same applies to the fourth embodiment.

### (Other Technical Ideas)

Technical ideas capable of being gained from the embodiments and the modifications described above will now be described.
[1] A continuous positive airway pressure apparatus comprising: a storage device configured to store setting data; and an execution device, wherein the execution device performs a storage process to store first identification data indicating a rewritable user in the storage device, an identification data acquiring process to acquire second identification data indicating a user associated with the setting data to be acquired in update in response to a request for the update of the setting data, a determination process to determine whether a specific condition is met, the specific condition including a requirement in which the first identification data is correlated with the second identification data in advance, and an update process to update the setting data stored in the storage device to the setting data associated with the second identification data if it is determined in the determination process that the specific condition is met.
[2] The continuous positive airway pressure apparatus described in [1], wherein the execution device performs an expiration date acquiring process to acquire expiration date data indicating an expiration date of the setting data to be acquired in update in response to a request for the update of the setting data, and wherein the specific condition includes a requirement in which a current date and time is before the expiration date indicated by the expiration date data and the first identification data is correlated with the second identification data in advance.
[3] The continuous positive airway pressure apparatus described in [2], wherein the execution device performs a warning process to issue a warning to a user of the continuous positive airway pressure apparatus if the current date and time is after the expiration date indicated by the expiration date data.
[4] The continuous positive airway pressure apparatus described in any of [1] to [3], wherein the execution device performs a confirmation process to confirm whether update of the setting data is required with a user of the continuous positive airway pressure apparatus if it is determined in the determination process that the specific condition is not met, and a forced update process to update the setting data stored in the storage device to the setting data associated with the second identification data regardless of the specific condition if the request for update of the setting data is accepted in the confirmation process.
[5] The continuous positive airway pressure apparatus described in any of [1] to [4], wherein, in response to a request for update of the setting data, the execution device performs a result transmitting process to transmit update result data indicating whether the setting data stored in the storage device is updated to an external device.
[6] The continuous positive airway pressure apparatus described in [5], wherein, if the execution device transmits the update result data indicating that the setting data is not updated in the result transmitting process and accepts a request for update of the setting data from the external device, the execution device performs a forced update process to update the setting data stored in the storage device to the setting data associated with the second identification data regardless of the specific condition.
[7] An update system of continuous positive airway pressure setting data, the update system comprising: a continuous positive airway pressure apparatus including a device-side storage device storing setting data and a device-side execution device; and a server including a server-side storage device storing the setting data and a server-side execution device updating the setting data in the continuous positive airway pressure apparatus, wherein the device-side execution device performs a storage process to store first identification data indicating a rewritable user in the device-side storage device, and wherein the server-side execution device performs a first identification data acquiring process to acquire the first identification data stored in the device-side storage device in response to a request for update of the setting data in the continuous positive airway pressure apparatus, a second identification data acquiring process to acquire second identification data indicating a user associated with the setting data stored in the server-side storage device in response to a request for update of the setting data in the continuous positive airway pressure apparatus, a determination process to determine whether a specific condition is met, the specific condition including a requirement in which the first identification data is correlated with the second identification data in advance, and an update process to transmit the setting data associated with the second identification data to the continuous positive airway pressure apparatus to update the setting data stored in the device-side storage device to the setting data associated with the second identification data if it is determined in the determination process that the specific condition is met.
[8] An update program of continuous positive airway pressure setting data, the update program being applied to a continuous positive airway pressure apparatus including a storage device storing setting data and an execution device, the update program causing the execution device to perform: a storage process to store first identification data indicating a rewritable user in the storage device, an identification data acquiring process to acquire second identification data indicating a user associated with the setting data to be acquired in update in response to a request for the update of the setting data, a determination process to determine whether a specific condition is met, the specific condition including a requirement in which the first identification data is correlated with the second identification data in advance, and an update process to update the setting data stored in the storage device to the setting data associated with the second identification data if it is determined in the determination process that the specific condition is met.
[9] An update method of continuous positive airway pressure setting data, the update method being applied to a continuous positive airway pressure apparatus including a storage device storing setting data and an execution device, wherein the execution device performs a storage process to store first identification data indicating a rewritable user in the storage device, an identification data acquiring process to acquire second identification data indicating a user associated with the setting data to be acquired in update in response to a request for the update of the setting data, a determination process to determine whether a specific condition is met, the specific condition including a requirement in which the first identification data is correlated with the second identification data in advance, and an update process to update the setting data stored in the storage device to the setting data associated with the second identification data if it is determined in the determination process that the specific condition is met.
[10] An update program of continuous positive airway pressure setting data, the update program being applied to an update system including a continuous positive airway pressure apparatus including a device-side storage device storing setting data and a device-side execution device; and a server including a server-side storage device storing the setting data and a server-side execution device updating the setting data in the continuous positive airway pressure apparatus, wherein the device-side execution device performs a storage process to store first identification data indicating a rewritable user in the device-side storage device, and wherein the update program causes the server-side execution device to perform a first identification data acquiring process to acquire the first identification data stored in the device-side storage device in response to a request for update of the setting data in the continuous positive airway pressure apparatus, a second identification data acquiring process to acquire second identification data indicating a user associated with the setting data stored in the server-side storage device in response to a request for update of the setting data in the continuous positive airway pressure apparatus, a determination process to determine whether a specific condition is met, the specific condition including a requirement in which the first identification data is correlated with the second identification data in advance, and an update process to transmit the setting data associated with the second identification data to the continuous positive airway pressure apparatus to update the setting data stored in the device-side storage device to the setting data associated with the second identification data if it is determined in the determination process that the specific condition is met.
[11] An update method of continuous positive airway pressure setting data, the update method being applied to an update system including a continuous positive airway pressure apparatus including a device-side storage device storing setting data and a device-side execution device; and a server including a server-side storage device storing the setting data and a server-side execution device updating the setting data in the continuous positive airway pressure apparatus, wherein the device-side execution device performs a storage process to store first identification data indicating a rewritable user in the device-side storage device, and wherein the server-side execution device performs a first identification data acquiring process to acquire the first identification data stored in the device-side storage device in response to a request for update of the setting data in the continuous positive airway pressure apparatus, a second identification data acquiring process to acquire second identification data indicating a user associated with the setting data stored in the server-side storage device in response to a request for update of the setting data in the continuous positive airway pressure apparatus, a determination process to determine whether a specific condition is met, the specific condition including a requirement in which the first identification data is correlated with the second identification data in advance, and an update process to transmit the setting data associated with the second identification data to the continuous positive airway pressure apparatus to update the setting data stored in the device-side storage device to the setting data associated with the second identification data if it is determined in the determination process that the specific condition is met.

What is claimed is:

1. A continuous positive airway pressure apparatus comprising:
a storage device configured to store setting data;
an execution device,
wherein the execution device is configured to:
reset the setting data in response to a reset request,
store a reset timing at which the setting data has been reset in the storage device as reset timing data,
acquire creation timing data indicating a creation timing of the setting data to be acquired in update in response to a request for the update of the setting data,
determine whether a specific condition is met, the specific condition including a requirement in which the reset timing indicated by the reset timing data is before the creation timing indicated by the creation timing data, and
update the setting data stored in the storage device to the setting data associated with the creation timing data if it is determined in the determination process that the specific condition is met.

2. The continuous positive airway pressure apparatus according to claim 1,
wherein the execution device is further configured to acquire expiration date data indicating an expiration date of the setting data to be acquired in update in response to a request for the update of the setting data, and
wherein the specific condition includes a requirement in which a current date and time is before the expiration date indicated by the expiration date data and the reset timing indicated by the reset timing data is before the creation timing indicated by the creation timing data.

3. The continuous positive airway pressure apparatus according to claim 2, wherein the execution device is further configured to issue a warning to a user of the continuous positive airway pressure apparatus if the current date and time is after the expiration date indicated by the expiration date data.

4. The continuous positive airway pressure apparatus according to claim 1, wherein the execution device is further configured to:
confirm whether update of the setting data is required with a user of the continuous positive airway pressure apparatus if it is determined that the specific condition is not met, and
update the setting data stored in the storage device to the setting data associated with the creation timing data regardless of the specific condition if the request for update of the setting data is accepted in the confirmation process.

5. The continuous positive airway pressure apparatus according to claim 1, wherein, in response to a request for update of the setting data, the execution device is further configured to transmit update result data indicating whether the setting data stored in the storage device is updated to an external device.

6. The continuous positive airway pressure apparatus according to claim 5, wherein, if the execution device transmits the update result data indicating that the setting data is not updated and accepts a request for update of the setting data from the external device, the execution device is further configured to update the setting data stored in the storage device to the setting data associated with the creation timing data regardless of the specific condition.

7. An update system of continuous positive airway pressure setting data, the update system comprising:
a continuous positive airway pressure apparatus including a device-side storage device configured to store setting data and a device-side execution device; and
a server including a server-side storage device configured to store the setting data and a server-side execution device configured to update the setting data in the continuous positive airway pressure apparatus,
wherein the device-side execution device is configured to:
reset the setting data in response to a reset request, and
store a reset timing at which the setting data has been reset in the device-side storage device as reset timing data, and
wherein the server-side execution device is configured to:
acquire creation timing data indicating a creation timing of the setting data stored in the server-side storage device in response to a request for update of the setting data in the continuous positive airway pressure apparatus,
acquire the reset timing data stored in the device-side storage device in response to a request for update of the setting data in the continuous positive airway pressure apparatus,
determine whether a specific condition is met, the specific condition including a requirement in which the reset timing indicated by the reset timing data is before the creation timing indicated by the creation timing data, and
transmit the setting data associated with the creation timing data to the continuous positive airway pressure apparatus to update the setting data stored in the device-side storage device to the setting data associated with the creation timing data if it is determined in the determination process that the specific condition is met.

8. An update method of continuous positive airway pressure setting data, the update method being applied to a continuous positive airway pressure apparatus including a storage device storing setting data and an execution device, the method comprising:
resetting the setting data in response to a reset request,
storing a reset timing at which the setting data has been reset in the storage device as reset timing data,
acquiring creation timing data indicating a creation timing of the setting data to be acquired in update in response to a request for the update of the setting data,
determining whether a specific condition is met, the specific condition including a requirement in which the reset timing indicated by the reset timing data is before the creation timing indicated by the creation timing data, and
updating the setting data stored in the storage device to the setting data associated with the creation timing data in response to determining that the specific condition is met.
